(19) United States
(12) Patent Application Publication
(10) Pub. No.: US 2024/0240165 A1
(43) Pub. Date: Jul. 18, 2024

(54) SYNTHETIC CAS PROTEINS

(71) Applicant: ASOCIACIÓN CENTRO DE INVESTIGACIÓN COOPERATIVA EN NANOCIENCIAS "CIC NANOGUNE", San Sebastian, Guipuzcoa (ES)

(72) Inventors: Raúl PEREZ-JIMENEZ, San Sebastian (ES); Borja ALONSO-LERMA, San Sebastian (ES)

(73) Assignee: ASOCIACIÓN CENTRO DE INVESTIGACIÓN COOPERATIVA EN NANOCIENCIAS "CIC NANOGUNE", San Sebastian, Guipuzcoa (ES)

(21) Appl. No.: 18/563,699

(22) PCT Filed: May 25, 2022
(86) PCT No.: PCT/EP2022/064307

§ 371 Date: Nov. 22, 2023

(30) Foreign Application Priority Data
May 25, 2021 (EP) 21382474.1
May 28, 2021 (GB) 2107671.6
Mar. 30, 2022 (EP) 22165690.3

(51) Int. Cl. C12N 9/22; C12N 15/11; C12N 15/90; G16B 10/00

(52) U.S. Cl. CPC C12N 9/22; C12N 15/11; C12N 15/907; G16B 10/00; C12N 2310/20; C12N 2800/80

(57) ABSTRACT

The use of phylogenetic ancestral sequence reconstruction to generate new Cas enzymes with improved capabilities. By this strategy, ancestral variants of Cas9 proteins of currently existing species have been obtained, which can exhibit nickase activity separate from endonuclease activity, and relaxed, if not abolished, PAM requirement. Ability to use tracrRNA components of Cas9 gRNAs from a wide variety of existing bacterial species has also been observed.

### FIELD OF THE INVENTION

The present invention relates to methods of obtaining Cas proteins suitable for use as single effector CRISPR system-associated nucleases, i.e., class II Cas proteins, which are not isolatable from recognized microbial sources. To do so, the invention provides reconstructed ancestral sequences derived by evolutionary tracing from a phylogenetic tree compiled using Cas protein sequences of existing species. Such reconstructed proteins are thus synthetic proteins in the sense that they are not isolatable from modern day sources but can be utilized in the same way as naturally-occurring Cas proteins in class II CRISPR systems which are now widely used for gene-editing. The Inventors have coined the term “ancestral Cas” or “AnCas” for such reconstructed sequences. This route to novel Cas proteins has been found to beneficially add to the diversity of Cas proteins available for gene-editing as regards useful properties, including relaxation of the protospacer adjacent motif (PAM) requirement of type II proteins compared with the most commonly used type II Cas protein, Streptococcus pyogenes (SpyCas9).

### BACKGROUND OF THE INVENTION

The native CRISPR-Cas systems provide immunity to prokaryotes responding to invading nucleic acids from infectious genetic elements. Cas proteins guided by CRISPR-encoded RNA molecules (gRNAs) recognize specific regions of the foreign genome and cleave it for inactivation. Since the first CRISPR-Cas9 system was repurposed as a gene-editing tool, such CRISPR systems, and other class II CRISPR-Cas systems, have revolutionized the field of genome engineering. Nevertheless, CRISPR is not ready for implementation as a therapeutic tool due to limitations such as generation of unwanted mutations at similar loci, production of multiple alleles leading to genetic mosaicism, low efficiency and possible induction in the host of an immune response. A study found that blood samples from human donors showed a high percentage with antibodies to SpyCas9 and Cas9 derived from Staphylococcus aureus (Charlesworth et al., 2019. Nature Med. 25(2):249-254).

The number and diversity of known CRISPR-Cas systems has dramatically increased since the first disclosure of in vitro DNA-editing studies with a CRISPR-Cas9 system in 2012. The distinguishing feature of class II systems is that the nuclease effector of the complex consists of a single, multi-domain protein, as exemplified by Cas9 utilizing type II systems. Target recognition is accomplished with structural non-coding RNAs that, through base-pairing, guide the Cas protein to its target nucleic acid sequence site for endonuclease cleavage action. In addition to guide RNA (gRNA) recognition, a sequence motif, termed the protospacer adjacent motif (PAM), is required for the initiation of Cas-guide RNA target binding and cleavage. This is important for distinguishing self from non-self in native anti-viral defense systems. However, it is less helpful for some desired applications arising from repurposing of class II CRISPR-Cas systems as a genetic tool.

Bacterial Cas9 proteins were the first studied Cas proteins and Spycas9 remains the most extensively studied Cas9 and much used for gene-editing. Such proteins are characterized as type II Cas9 proteins by containing two nuclease domains, both an HNH-like and RuvC-like nuclease domain, and associated catalytic residues required for double-strand DNA endonuclease cleavage resulting in blunt ends. Since 2012, Cas endonucleases have been isolated from many different bacteria and archaea. The latest classification of class II Cas nucleases includes 3 types and 17 sub-types as reviewed in Makarova et al., 2020 (Nature Rev Microbiol. 18(2):67-83). Thus, class II CRISPR-Cas systems currently include types II, V and VI systems with type VI systems being the first and so far, only variety of CRISPR-Cas systems that exclusively cleave RNA.

The type V systems fundamentally differ from type II systems by the domain architecture of their effector Cas proteins. Whilst type II effectors (Cas9 nucleases) contain two nuclease domains that are each responsible for the cleavage of one strand of the target DNA, with the HNH nuclease inserted inside the RuvC-like nuclease domain sequence, the type V effectors (Cas12 nucleases) by contrast only contain a RuvC-like domain that cleaves both strands. Type VI effectors (Cas13 nucleases) are unrelated to the effectors of type II and type V, as they contain two HEPN domains and apparently target transcripts of invading DNA genomes in their native environment. Cas13 proteins also display collateral, non-specific RNase activity that is triggered by target recognition.

Amongst type V variants, a number with smaller RuvC-like domains are currently classified as sub-type V-U effectors. These show high sequence similarity to TnpB proteins (predicted RuvC-like nucleases) encoded by IS605-like transposons and are thought to be intermediates on the evolutionary path from TnpB to fully fledged type V effectors. CRISPR-Cas systems evolved from different groups of TnpB on multiple, independent occasions, as has been shown by phylogenetic analysis of the TnpB family. Analysis of the interference activity of four subtype V-U effectors has more recently resulted in one such variant being upgraded to a separate subtype V-F. The subtype V-F effector, Cas12f (originally denoted Cas14), has been shown to cleave single-stranded DNA (ssDNA). However, phylogenetic analysis of type V Cas enzymes has only been used as a means of classifying isolated naturally-occurring such nucleases with a single RuvC-like nuclease domain.

Despite the diversity of known naturally-occurring class II CRISPR-Cas systems, recently supplemented by the extensive mining of natural sources for CRISPR-Cas9 orthologs by Gasiunas et al., 2020 (Nature Com. 11(1):5512), still further diversity is desired to ease various gene-modification desires, especially in the therapeutic field. SpyCas9 has been widely-adapted for genome editing and as a fusion enzyme for transcriptional control, epigenome-editing, base-editing and prime-editing. Despite its versatility, SpyCas9 is still limited for certain such applications by its “NGG” PAM recognition requirement. Attempts have been made to relax this requirement not only by mining for new orthologs, but by utilizing directed evolution techniques, e.g., random mutagenesis of the PAM interaction domain (PID) with selection, and by structure-guided mutagenesis as reviewed by Collias & Beisel, 2021 (Nature Com. 12(1): 555). Walton et al., 2020 (Science. 368(6488):290-296), by application of structure-guided mutagenesis, achieved the SpRY nuclease variant which recognizes a consensus NR PAM sequence (with R being A or G) and, with lesser efficacy, a RY PAM sequence (with Y being C or T). This is the most relaxed PAM requirement reported for a Cas9 variant to date.

### SUMMARY OF INVENTION

As indicated above, the inventors in this instance have adopted a novel approach to expand the available toolset of RNA-programmable CRISPR-associated nucleases. This approach referred to as Phylogenetic Ancestral Sequence Reconstruction has been used to generate variants of bacterial Cas9 predicted to have been present in organisms that lived billions of years ago. Ancestral enzymes have greater stability and efficiency, exhibit chemical promiscuity and are more versatile than their modern descendants. Furthermore, a benefit of looking to ancestral enzymes for gene therapy is that the host's pre-existing immunity against these proteins can be potentially dismissed. The inventors have designed and tested, for example, ancestral Firmicutes, Bacilli and Streptococci Cas9 forms. They show high level of expression, non-specific tracrRNA binding, and high efficiency gene editing in cells of the human HEK293T cell line.

Whilst the invention has been founded on use of phylogenetic information for a diverse population of Cas9 enzymes from the phylum Firmicutes and within the bacterial classes of Clostridia and Bacilli, including many species of Streptococcus encompassing for example, Streptococcus pyogenes, plus some Cas9 sequences from the phylum of Actinobacteria, it will be appreciated that the same approach may be employed to obtain ancestral versions of Cas single nuclease effectors of other classification types, e.g. an ancestral Type V or Type VI Cas enzyme. The ancestral version may be of the same type but of a different sub-type. It may be assigned a novel sub-type from any sub-type of the current classification as set out in Makarova et al. ibid.

Thus, in one aspect the present invention provides a method of phylogenetic ancestral reconstruction for obtaining a functional, single effector Cas protein nuclease (commonly referred to as Class II Cas protein), e.g., a functional Cas9 variant, comprising:
(a) providing a phylogenetic tree from sequence analysis of a population of Cas sequences comprising naturally-occurring single effector Cas nuclease sequences of the same classification type, e.g., a population of Type II Cas9 sequences, and derived from a plurality of existing species, preferably of more than one genus, still more preferably of more than one class and possibly spanning more than one phylum;
(b) selecting an ancestral variant sequence by tracing back an evolutionary route from the phylogenetic tree, wherein the highest probability amino acid for each amino acid of the selected ancestral variant is determined, and
(c) producing said variant, wherein said variant is capable of exhibiting Cas protein endonuclease and/or nickase activity.

It will be appreciated that the starting population of Cas sequences for provision of the phylogenetic tree of step (a) may include one or more predetermined ancestral variant sequences obtained by prior application of such a method.

Computer-implemented methods for compiling phylogenetic trees by protein sequence alignment of protein orthologs are well-known. Described further herein is use of computer-implemented methods whereby evolutionary routes can be compiled enabling ancestral variants of naturally-occurring Cas proteins to be predicted and reconstructed from many millions of years ago. The inventors report for the first time the “resurrection” of Cas9 enzymes as old as 2-3 Bys showing high production level as well as high efficiency of targeting and editing of DNA.

In effect, computer-implementation of step (b) may comprise:
(i) compiling sequences of ancestral variants which are each just ancestral variants for a plurality of species' sequences forming a proportion of the sequences of the same genus, e.g., the Streptococcus genus, preferably further
(ii) using the sequences attained in (i) to compile one or more ancestor variant sequences which are assigned as a genus ancestor, e.g., an ancestral sequence which is a compiled ancestral sequence just for all or at least a large proportion of available Streptococci Cas sequences and/or one or more ancestor variant sequences which are assigned as a class ancestor able to trace back to sequences of starting species of a plurality of genera, e.g. an ancestral sequence which is compiled as an ancestor sequence just for all or at least a large proportion of available Bacilli sequences spanning a plurality of genera, preferably further
(iii) compiling at least one inter-class ancestor sequence able to trace back to starting species of more than one class. Such an ancestral variant may be a preferred selection for production, but a variety of ancestral variants thus compiled may be found to have beneficial properties.

One such evolutionary route map is shown in FIG. 1 leading to compilation of such an inter-class ancestral variant sequence (or common phylum ancestor sequence) starting from a population of Cas9 sequences as noted above, including Cas9 sequences of existing bacterial species of both the Bacilli and Clostridia classes. It will be noted that the starting sequences of bacterial species of the Bacillus class include many known Cas9 sequences of Streptococci including SpyCas9 (28 in number). Use of such a diverse population of starting sequences, including Cas9 sequences from a diverse range of bacteria belonging to the Bacillus class, including a substantial number of existing Streptococci Cas9 sequences, e.g., 25 or more, will be recognized as highly desirable for such evolutionary map construction.

The production of step (c) will normally be by providing a nucleic acid sequence for expression in a suitable host cell, e.g., E. coli. The coding sequence may be codon-optimized.

The exemplification illustrates how desired cleavage activity may be tested for even in the absence of knowledge of any PAM requirement. Desirably where such activity is observed initially by in vitro test, it will be maintained in further testing in human cells. For example, where the selected ancestral variant has been attained starting from a population of Cas9 sequences from a plurality of existing species, the activity of the selected ancestral variant may be tested under conditions in vitro and in a human cell line known to be suitable for endonuclease activity of a Cas9 sequence from an existing species, e.g., SpyCas9. For such testing in human cells, human codon-optimized sequences for the Cas enzymes will desirably be employed in expression vectors suitable for Cas protein expression in the chosen cells.

Where the initially selected variant is a Cas endonuclease, it may be subsequently converted to a nickase or converted to a deadCas (dcas) in known manner for amino acid mutagenesis of relevant nuclease catalytic sites and/or fused to a non-nuclease effector. For example, it is well known how to inactivate one or both nuclease sites of a Cas9 endonuclease and link a Cas9 enzyme to another effector, e.g., an enzyme for base-editing or prime-editing or a transcriptional or epigenetic regulator.

Also encompassed by the invention are novel Cas enzymes obtained by an ancestral reconstruction method as described above and nucleic acid sequences encoding the same, e.g., provided in an expression vector for expression in a host cell. Within the scope of the present invention, the Cas nucleases or Cas nuclease variants described herein are hereby interchangeably referred to as “ancestral Cas” or “AnCas”.

By ancestral sequence reconstruction as now taught, of particular interest is the attainment of AnCas variant enzymes which, compared to SpyCas9 under conditions for linearization of a dsDNA plasmid target by SpyCas as a reference nuclease, will exhibit time separable nickase and endonuclease activity reflected by a higher ratio of nicked template to linearized template. That is to say that the AnCas variant enzymes described herein may produce a greater percentage of nicked plasmid DNA template compared to SpyCas9 under the same conditions or may produce a lower percentage of double stranded breaks in a plasmid DNA template compared to SpyCas9 under the same conditions. Indeed, as is well-known, SpyCas9 is not recognized as a nickase enzyme under commonly employed conditions of use except when one nuclease site is removed. In contrast, now provided are AnCas enzymes obtained as Cas9 ancestral variants having a ratio of linearized DNA plasmid template to nicked DNA plasmid template of between at least 2.3:1 to at least 1:4 under conditions whereby SpyCas9 results in a ratio of linearized DNA plasmid target to nicked DNA plasmid template of at least 4:1. That is to say after 30 minutes AnCas enzymes obtained in accordance with the invention such as LFCA, LBCA and LSCA may nick at least 30% of the DNA template up to at least 70%, e.g. about 80%, of the DNA template whereas under the same conditions SpyCas9 nicks about 10% of the DNA template in the same amount of time (see FIG. 11). Thus, in other words, the AnCas nuclease has a higher nick rate and lower linearization rate on a dsDNA plasmid target under conditions whereby SpyCas9 results in substantially exclusively linearization or almost exclusively linearization while the AnCas nuclease and variants of interest provide under the same conditions observable nicked target.

This may be combined in the AnCas enzyme with relaxed PAM requirement compared with SpyCas9; indeed, no observable 3 nucleotide or up to 7 nucleotide specific PAM requirement under conditions where SpyCas9 maintains its recognized 3 nucleotide PAM requirement of NGG, e.g., TGG, to exhibit significant DNA cleavage activity.

Moreover, such relaxed PAM requirement has been observed to be combined with highly flexible gRNA use. Thus, as reported herein an AnCas has been found to be capable of utilizing a sgRNA with a Cas9 tracrRNA component corresponding to that of any of a wide variety of existing bacterial species possessing a Cas9 ortholog. Thus, the targeting sequence may be varied but the sgRNA otherwise resembles a sgRNA as may be employed with a plurality of known Cas9 orthologs. Such non-specific tracrRNA use is not a property which has been reported previously for any known Cas9 ortholog.

Such beneficial characteristics have also been attained in combination with ability to cleave single-stranded DNA and single-stranded RNA. Again, this is a distinguishing characteristic from SpyCas9.

By way of example of such an AnCas, now provided is a highly advantageous ancestral of existing Firmicute Cas9 enzymes including SpyCas9, designated by the inventors the Last Firmicute Common Ancestor (LFCA); see FIG. 1, node 63 of FIG. 9 and SEQ ID NO: 1.

LFCA
SEQ ID NO: 1
MKKDYSIGLDIGTNSVGWAVTDDNYNLVRKKMKVLGNTDK
KSIGKALWGVRLFDAAETAEERRMHRTTRRRYTRRRQRID
LLQEIFQEEISKVDPSFFIRLNESRLHPEDKTDDRHPLFG
DNETDKDYHKQYPTIYHLRKHLMESDEKHDIRLVYLALHH
IIKYRGHFLIEGDLNSENTDVEELFKQLVQVENDTFEEEH
LSEEAIDIEEILTDKKSRSTRAKEVVKLFGSEKKQACISA
LIKLIVGLKGNLKKVFGDAEDTSIHFSKDNYEEDLEAIRD
IIPDEYADLFEAIKALYDAIVLSGILGGSTSNTKAKVSAS
MIARYEQHQKDLKQLKQFVKEHLPEKYNEIFSDNTKNGYS
AYIEGGTSQEDFYKYLKKILKELEEAEYLLEEIENENFLP
KQRTSDNGVIPYQIHLEELRAILKNOGKYYPFLKENAEKI
ESILTFRIPYYVGPLARGNGRFAWMIRKKDGKITPWNFDE
VVDKEKSAEEFIERMTNNCTYLPGENVLPKNSLLYEKFTV
LNELNNVRLTTDKGKTRRFSAEQKQEIFDDLFRKNRKVTK
KKLEDYLKREYEEFDSADISGIDGEFKSSLGSYHDFCKIV
VKGNSLDEEDYKDIIEEIIKWLTVFEDRKMLRRRLEKYSE
ILTEEQIKKICKRHYTGWGRLSRKLLTGIRDKETGKSIID
VLRETDSSNRNFMQLLSDEDLSFKEEIEQANAEAEGENLH
EIVEDLPGSPAIKRGILQALKIVDEIVKVMGHEPKNIFVE
MARENQKTGRGRRSTKKRLKRLQEALKNLGSNLLKELPRD
DNELRNDRLFLYYTQMGKCMYTGEPIDLDDLSNYDIDHII
PQSFIKDDSLDNRVLVSREENARKTDNFPSPEIRRKMKSF
WQMLLKAGLISKKKFDRLTRADRGDFTDDELAGFIARQLV
ETRQITKHVATLLKQRYPTEKDEEDKTIRNAKIVSVKANL
VSEFRQDFGLYKCREVNDYHHAHDAYLNAVVGNALLKKYP
QLAAEFIKGDYRKNNAREENKANAKMHFYSNIMNSFTSDV
KIADETGEIVWDKEKDIATVRKVMNYHQVLITRKVEEEKG
GFFDQTILSKGNSKKLIPLKKNLDPEKYGGYNSPTVAYSV
LVEYDIEKGKKKKLKTVKQLVGIPIRERAKLEKNPIKYLE
KKGYQNPKVDLLIKIPKNSLFELDGGRRRILAAAKELKNA
NQLVLPAEEYTLLDKVAKIIKKNNSESIEYVEEHLSEFDE
LLESLIDYSPKLALQDKNLEKIKEAFEQLNLADKKEVAKE
IINLLHCTATTANAALKFLGGSKNRMRYTSIKELLNASLI
HQSITGLYETRIDLGKLGED

By way of further example, also now provided is a highly advantageous ancestral variant of existing Bacilli Cas9 enzymes including SpyCas9, designated by the inventors the Last Bacilli Common Ancestor (LBCA); see FIG. 1, see node 70 of FIG. 9 and SEQ ID NO: 2. This AnCas illustrates a class ancestral variant being evolutionary traceable to the Cas sequences of a broad range of modern-day bacterial species of the Bacilli class, including Streptococci species.

LBCA
SEQ ID NO: 2
MKKDYSIGLDIGTNSVGWAVITDDYKVVRKKMKVLGNTDK
KSIKKNLWGVRLFDSGETAEDTRLKRTTRRRYTRRRNRIC
YLQEIFQEEMNKVDDSFFHRLDESFLVPEDKKYDRHPIFG
NLEEEVAYHEQYPTIYHLRKHLADSSEKADLRLVYLALAH
IIKYRGHFLIEGDLNTENTDVEELFKQFVQVYNQTFEEQH
LSDETIDVEEILTEKVSKSRRAENVLKLFPNEKKNGLFGQ
LIKLIVGLQGNFKKVFDLSEDAKLQFSKDTYEEDLENLLA
MIGDEYADLFLAAKNLYDAILLSGILTTTDNNTKAKLSAS
MIKRYEEHQKDLAQLKQFIKEHLPDKYNEIFSDSSKNGYA
GYIEGKTSQEDFYKYLKKILSKIDGAEYFLEKIEQENFLR
KQRTFDNGVIPHQIHLEELRAILRRQGKYYPFLKENQEKI
EQILTFRIPYYVGPLARGNSRFAWLTRKSDEPITPWNFDE
VVDKEKSAEAFIERMTNYDTYLPNEKVLPKHSLLYEKFTV
FNELTKVRYVTDRGKTQNFSAEQKQEIFDDLFKKNRKVTK
KKLENYLKKEYEYFDSPDITGIEDEFNASLGTYHDLLKIL
KSKDFLDDEENEEILEDIVKILTVFEDRKMIRKRLEKYSD
ILTEEQLKKLERRHYTGWGRLSRKLINGIRDKQSGKTILD
YLIDDDSSNRNFMQLINDDNLSFKEEIEKAQVIGETENLH
EIVQDLPGSPAIKKGILQSLKIVDELVKVMGHEPKNIVVE
MARENQTTSRGRRNSKQRLKRLEEALKNLGSNLLKEHPVD
NQQLONDRLYLYYLONGKDMYTGQELDIDNLSNYDIDHII
PQSFIKDNSIDNRVLVSSEENRGKSDNVPSKEVVRKMKSF
WQKLLNAGLISQRKFDNLTKAERGGLTEDDKAGFIKRQLV
ETRQITKHVANILDSRFNTEKDEEDNTIRNVKIITLKSNL
VSQFRKDFGLYKVREINDYHHAHDAYLNAVVGTALLKKYP
QLEPEFVYGDYRKNNAREENKATAKKHFYSNIMNFFASEV
KIADETGEIVWDKEKDIATVRKVLSYHQVNIVKKVEVQKG
GFSKETILPKGNSNKLIPRKNNWDPKKYGGFDSPTVAYSV
LVTYDIEKGKKKKLKTVKELVGITIMERSAFEKNPIAYLE
KKGYQNPQEDVLIKLPKYSLFELENGRRRMLASAKELQKG
NQMVLPAHLVTLLYHAKRIDKSNNSESLEYVEEHRNEFDE
LLDYIIDFSEKYILADKNLEKIKKLYEQNNEADIKELAKS
FINLLTFTAMGAPAAFKFFGETIDRKRYTSIKELLNATLI
HQSITGLYETRIDLGKLGED

By way of example, additionally now provided is a highly advantageous ancestral variant of SpyCas9, designated by the inventors the Last Streptococci Common Ancestor (LSCA); see FIG. 1, node 91 of FIG. 9 and SEQ ID NO: 3. This AnCas illustrates an AnCas deemed a common genus ancestor. It is a created common ancestor of all the starting Streptococci sequences listed in Table 1, including SpyCas9.

LSCA
SEQ ID NO: 3
MKKPYSIGLDIGTNSVGWAVITDDYKVPAKKMKVLGNTDK
QSIKKNLLGALLFDSGETAEATRLKRTARRRYTRRRNRIC
YLQEIFSEEMNKVDDSFFHRLDESFLVPEDKKYDRHPIFG
NLAEEVAYHEQYPTIYHLRKHLADSTEKADLRLVYLALAH
IIKFRGHFLIEGDLNAENTDVQKLFQQFVEVYNQTFEESH
LSEETIDVEEILTEKISKSRRLENLIKHFPNEKKNGLFGN
LIALILGLQPNFKTNFDLSEDAKLQFSKDTYEEDLENLLA
QIGDEYADLFLAAKNLYDAILLSGILTVTDNSTKAPLSAS
MIKRYEEHQKDLAQLKQFIKEHLPDKYNEIFSDKSKNGYA
GYIEGKTSQEDFYKYLKKILSKIDGAEYFLDKIDREDFLR
KQRTFDNGSIPHQIHLQELHAILRRQGEYYPFLKENQEKI
EKILTFRIPYYVGPLARGNSRFAWLTRKSDEKITPWNFDE
VVDKESSAEAFIERMTNYDTYLPNEKVLPKHSLLYETFTV
YNELTKVKYVTERGKTQFFSAEQKQEIFDHLFKKNRKVTK
KKLKDYLEKEFEEFDSVDITGVEDEFNASLGTYHDLLKIL
KDKDFLDDEENEEILEDIVLTLTLFEDREMIRKRLEKYSD
LFTKEQLKKLERRHYTGWGRLSRKLINGIRDKQSGKTILD
YLIDDGSSNRNFMQLINDDSLSFKEEIEKAQVIGETDNLH
EVVQDLAGSPAIKKGILQSLKIVDELVKVMGHNPENIVVE
MARENQTTNRGRRNSRQRLKRLEEALKNLGSNILKEHPVD
NQQLQNDRLYLYYLQNGKDMYTGEELDIDNLSQYDIDHII
PQSFIKDDSIDNRVLTSSEENRGKSDNVPSIEVVRKMKSF
WQKLLNAGLISQRKFDNLTKAERGGLTEDDKAGFIKRQLV
ETRQITKHVAQILDSRENTERDENDKRIRNVKIITLKSNL
VSQFRKDFGLYKVREINDYHHAHDAYLNAVVGTALLKKYP
KLEPEFVYGDYKKYNDRERGKATAKMFFYSNIMNFFKTEV
KLADETGEIVWDKEKDFATVRKVLSYPQVNIVKKVEVQTG
GFSKESILPKGNSDKLIPRKNNWDPKKYGGFDSPTVAYSV
LVVADVEKGKAKKLKTVKELVGITIMERSAFEKNPIAFLE
KKGYQNIQEDLIIKLPKYSLFELENGRRRLLASAKELQKG
NEMVLPAHLVTLLYHAKRIDKSNNSENLEYVEKHKNEFDE
LLDYIIDFSEKYILADKNLEKIKELYDQNDDADINELASS
FINLLTFTALGAPAAFKFFGETIDRKRYTSTKEVLNATLI
HQSITGLYETRIDLSKLGED

Also now provided are ancestral variants of SpyCas9, designated by the inventors the Last Pyogenic Common Ancestor (LPCA) and the Last Pyogenic-Dysgalactic Common Ancestor (LPDCA) evolutionary traceable to more than one Streptococci species including Streptococcus pyogenes; see nodes 92 and 95 in FIG. 9 respectively and SEQ ID Nos: 4 and 5.

LPCA
SEQ ID NO: 4
MKKPYSIGLDIGTNSVGWAVITDDYKVPAKKMKVLGNTDR
QSIKKNLIGALLFDSGETAEATRLKRTARRRYTRRKNRIC
YLQEIFSEEMAKVDDSFFHRLEESFLVPEDKKYDRHPIFG
NLADEVAYHENYPTIYHLRKKLADSTEKADLRLIYLALAH
IIKFRGHFLIEGDLNAENTDVQKLFHQLVDTYNQLFEEDQ
LDTETIDAKAILTAKISKSRRLENLISQIPGQKKNGLFGN
LIALSLGLTPNFKSNFDLSEDAKLQLSKDTYEEDLDNLLA
QIGDQYADLFLAAKNLSDAILLSDILTVNDESTKAPLSAS
MIKRYEEHQQDLALLKQLVKEQLPEKYKEIFSDKSKNGYA
GYIDGKTSQEEFYKYIKPILSKLDGAEEFLAKIDREDFLR
KQRTFDNGSIPHQIHLEELHAILRRQEEYYPFLKDNQEKI
EKILTFRIPYYVGPLARGNSRFAWLTRKSDEAITPWNFEE
VVDKEASAQAFIERMTNFDTYLPNEKVLPKHSLLYETFTV
YNELTKVKYVTEGMTKPFLSAEQKQAIVDLLFKKNRKVTV
KQLKEDYFKKIECFDSVDITGVEDRFNASLGTYHDLLKII
KDKDFLDNEENEDILEDIVLTLTLFEDREMIEKRLAKYAD
LFDKKVLKKLKRRHYTGWGRLSRKLINGIRDKQSGKTILD
FLKADGFANRNFMQLINDDSLSFKEEIEKAQVIGQTDSLH
EVVADLAGSPAIKKGILQTIKIVDELVKVMGHNPENIVIE
MARENQTTAQGIKNSRQRMKRLEEVLKKLGSNILKEHPVD
NTQLQNDRLYLYYLQNGKDMYTGQELDIDNLSQYDIDHII
PQSFIKDDSIDNKVLTSSEENRGKSDNVPSIEVVRKMKSY
WQKLLNAGLISQRKFDNLTKAERGGLTESDKAGFIKRQLV
ETRQITKHVAQILDSRENTERDENDKPIRNVKIITLKSKL
VSDFRKDFGLYKVREINDYHHAHDAYLNAVVGTALLKKYP
KLEPEFVYGDYKKYDDKERGKATAKMFFYSNIMNFFKTEV
KLANETGEIVWDKEKDFATVRKVLSYPQVNIVKKTEVQTG
GFSKESILPKGNSDKLIPRKNNWDPKKYGGFDSPTVAYSV
LVVAKVEKGKAKKLKTVKELVGITIMERSAFEKNPIAFLE
AKGYQDIQEDLIIKLPKYSLFELENGRRRLLASAKELQKG
NEMVLPAHLVTFLYHASRIDKSTSSENLEYVEQHKHEFDE
ILDYIIDFSERYILADKNLEKIKSLYNQNDDSDINELASS
FINLFTFTALGAPAAFKFFDATIDRKRYTSTKEVLNATLI
HQSITGLYETRIDLSQLGGD
LPDCA
SEQ ID NO: 5
MDKKYSIGLDIGTNSVGWAVITDDYKVPSKKFKVLGNTDR
HSIKKNLIGALLFDSGETAEATRLKRTARRRYTRRKNRIC
YLQEIFSNEMAKVDDSFFHRLEESFLVEEDKKHERHPIFG
NIVDEVAYHEKYPTIYHLRKKLADSTDKADLRLIYLALAH
MIKFRGHFLIEGDLNPDNSDVDKLFIQLVQTYNQLFEENP
INASGVDAKAILSARLSKSRRLENLIAQLPGEKKNGLFGN
LIALSLGLTPNFKSNFDLAEDAKLQLSKDTYDDDLDNLLA
QIGDQYADLFLAAKNLSDAILLSDILRVNSEITKAPLSAS
MIKRYDEHHQDLTLLKALVRQQLPEKYKEIFFDQSKNGYA
GYIDGGASQEEFYKFIKPILEKMDGTEELLAKLNREDLLR
KQRTFDNGSIPHQIHLGELHAILRRQEDFYPFLKDNREKI
EKILTFRIPYYVGPLARGNSRFAWMTRKSEETITPWNFEE
VVDKGASAQSFIERMTNFDKNLPNEKVLPKHSLLYEYFTV
YNELTKVKYVTEGMRKPFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRHYTGWGRLSRKLINGIRDKQSGKTILD
FLKSDGFANRNFMQLIHDDSLTFKEEIQKAQVSGQGDSLH
EQIANLAGSPAIKKGILQTVKVVDELVKVMGHKPENIVIE
MARENQTTQKGQKNSRERMKRIEEGIKELGSQILKEHPVE
NTQLQNEKLYLYYLONGRDMYVDQELDINRLSDYDVDHIV
PQSFIKDDSIDNKVLTRSDKNRGKSDNVPSEEVVKKMKNY
WRQLLNAKLITQRKFDNLTKAERGGLSELDKAGFIKRQLV
ETRQITKHVAQILDSRMNTKYDENDKLIREVKVITLKSKL
VSDFRKDFQFYKVREINNYHHAHDAYLNAVVGTALIKKYP
KLESEFVYGDYKVYDEQEIGKATAKRFFYSNIMNFFKTEI
TLANETGEIVWDKGRDFATVRKVLSMPQVNIVKKTEVQTG
GFSKESILPKRNSDKLIARKKDWDPKKYGGFDSPTVAYSV
LVVAKVEKGKAKKLKSVKELVGITIMERSSFEKNPIDFLE
AKGYKDVQKDLIIKLPKYSLFELENGRRRMLASAGELQKG
NEMVLPAKLVTFLYHASHIEKSKSPENNAYVEQHKHDLDE
ILEYISEFSKRYILADKNLSKVKSLFNKHEDSSISELASS
IINLFTLTSLGAPAAFKFLDTTIDRKRYTSTKEVLDATLI
HQSITGLYETRIDLSQLGGD

All of LFCA Cas, LBCA Cas and LSCA Cas have been shown to exhibit a higher nick rate and lower endonuclease (double stranded cleavage) rate than SpyCas9 under conditions suitable for SpyCas9 dsDNA cleavage. The ratio of these activities has been found to be a function of ancestral age with LFCA Cas having the highest ratio observed to date.

All of LFCA Cas, LBCA Cas and LSCA Cas also exemplify the utility of the ancestral reconstruction strategy of the invention to attain Cas9 variants with relaxation of the PAM requirement as noted above. Such AnCas nucleases may thus be deemed PAMless.

All of LFCA Cas, LBCA Cas and LSCA Cas have additionally been shown to be capable of cleaving single-stranded DNA as shown by studies reported herein. LFCA and LBCA Cas have also been shown to be capable of cleaving single-stranded RNA as shown by studies reported herein.

Finally, all of LFCA Cas, LBCA Cas and LSCA Cas have additionally been shown to trigger only weak responses to anti-Cas9 antibodies, as shown by studies reported herein. This feature may be of particular interest for in vivo applications.

The invention is described further below with reference to the following figures, and in the appended claims.

### BRIEF DESCRIPTION OF THE FIGURES

FIG. 1 illustrates ancestral Cas9 reconstruction and characterization. Shown is a phylogenetic tree of Cas9 enzymes from the Clostridia and Bacilli classes of the phylum Firmicutes, plus Cas9 enzymes from some Actinobacteria. The evolutionary route from Last Firmicute Common Ancestor (LFCA; SEQ ID NO 1) following the ancestors of Bacilli (LBCA; SEQ ID NO 2), Streptococci (LSCA; SEQ ID NO 3) and several streptococcus species ancestors to modern S. pyogenes (LPCA; SEQ ID NO 4 and LPCDA; SEQ ID NO 5) is indicated by the white dashed arrow. The sequence relationships are also presented in the simplified cladogram of FIG. 9.

FIGS. 2a-c illustrate testing of AnCas endonuclease activity as exemplified by testing of LFCA. FIG. 2a shows a DNA library containing 7 random nucleotides right after a target DNA; these 7 N represent all possible PAM sequences. FIG. 2b shows Cas9 activity assay comparing LFCA with SpyCas9. LFCA Cas cuts the PCR target amplified from the DNA library, producing two fragments with the expected sizes. FIG. 2c shows Cas9 activity assay using the S. pyogenes PAM sequence. LFCA is able to recognize the NGG PAM sequence as SpyCas9 does.

FIGS. 3a-d illustrate demonstrating nicking and endonuclease activity of LFCA. FIG. 3a shows a DNA plasmid containing a TGG PAM sequence after the DNA target. Cas9 can cut one or both strands of the DNA. FIG. 3b shows a 1% agarose gel of the DNA plasmid after 1 hour contact with 30 nM Cas9 resulting in endonuclease activity. LFCA under the same conditions shows nicking activity after 10 minutes of incubation and double strand cleavage after 1 hour. In contrast, SpyCas9 exhibits mostly double strand cutting activity. FIG. 3c shows total cleavage expressed in % (nicking and endonuclease activity) from both LFCA and SpyCas9 as a function of time. FIG. 3d shows cleavage expressed in % with a distinction made between nicking and endonuclease activity. LFCA cuts one strand of DNA and, after 1 hour of incubation, starts to cleave the other strand. SpyCas9 has mostly endonuclease activity (i.e., cuts both strands).

FIGS. 4a-b illustrate PAM determination for LFCA. FIG. 4a shows the PAM wheel from LFCA PAM assessment with provision of 3 nucleotide PAMs. LFCA does not show specificity of recognition for any particular PAM with 3 nucleotides. Similar results were obtained with 7 nucleotide PAMs. FIG. 4b shows in vitro cleavage assay results of plasmid DNA with different PAM sequences comparing both LFCA and SpyCas9. LFCA (10 nM) nicked all the plasmids with different PAMs within only 10 minutes of reaction. In contrast, SpyCas9 cut almost 100% of the plasmid containing its canonical PAM sequence, TGG, and showed low or no activity with other PAM sequences.

FIGS. 5a-d show thermal and pH stability testing of LFCA. FIG. 5a shows total Cas enzyme activity assay using plasmid DNA with a TGG PAM for 1 hour with 30 nM of Cas enzyme at pH 7.9 and different temperatures ranging from 4° C. to 60° C. LFCA shows higher activity than SpyCas9 at 4° C. and from 53-60° C. FIG. 5b shows nicking and endonuclease activity of both Cas enzymes at different temperatures. FIG. 5c shows total Cas enzyme activity assay using plasmid DNA with a TGG PAM for 1 hour and with 30 nM of Cas enzyme at 37° C. and different pH ranging from 4 to 9.5. LFCA showed higher activity at acidic pH (4-5.5) in comparison with SpyCas9. FIG. 5d shows nicking and endonuclease activity of both Cas enzymes at different pH.

FIGS. 6a-f illustrate a comparison of LFCA and SpyCas9 genome-editing in HEK293T cells. FIG. 6a shows humanized LFCA and SpyCas9 coding sequences cloned in expression plasmid pCDNA 3.1 for transfection with gRNA into HEK293T cells for targeting the AAVS1 locus. FIG. 6b shows an immunofluorescent image from cells transformed with either Cas enzyme. Cells expressing the hCas coding sequence are stained in orange, the nucleus is stained with DAPI. FIG. 6c shows results of a T7 assay for Cas enzyme activity. PCR products from cells transformed with gRNA and hCas coding sequence were incubated with T7E1 to measure indel formation. FIG. 6d shows hCas9, gRNA and donor DNA carrying eGFP gene transfected into HEK293T cells for knock-in of eGFP into the AAVS1 locus. FIG. 6e shows confocal microscopy images of cells expressing eGFP. FIG. 6f shows relative fluorescent measured in the images from cells expressing eGFP after hCas enzyme transfection.

FIG. 7 illustrates a comparison of LFCA and SpyCas9 knock-in in HEK293T cells targeting a TTC PAM. Also shown is an electrophoresis gel with the extracted gDNA from the cells and amplified locus. The bands with the expected size in all the samples are seen on the gel apart from the TTC PAM targeted with Spy Cas9.

FIG. 8 shows agarose gel test results illustrating the ability of LFCA to use a sgRNA in which the targeting sequence is linked to a tracRNA component corresponding to the Cas9 tracrRNA component of one of a wide variety of existing bacterial species having a Cas9 ortholog.

FIG. 9 provides a cladogram constructed using the sequences listed in Table 1. Each node represents an ancestral state with a sequence shown in the sequence listing.

FIGS. 10a-c show nicking and endonuclease Cas9 activity of LFCA, LBCA and LSCA in comparison to SpyCas9. FIG. 10a shows total cleavage (both nicking and endonuclease activity) for LFCA, LBCA, LSCA and SpyCas9. All the Cas9 enzymes reached total cleavage within around 10 minutes of incubation. FIG. 10b shows plasmid linearization rate of LFCA, LBCA, LSCA and SpyCas9. FIG. 10c shows nick rate of LFCA, LBCA, LSCA and SpyCas9.

FIG. 11 shows the percentage of DNA template with a double stranded break (DSB), i.e., percentage of linearized template after 30 minutes of incubation with the respective Cas nuclease and percentage of nicked DNA template after 30 minutes of incubation with the respective Cas nuclease. LFCA, LBCA, and LSCA produce a lower amount of linearized DNA template (i.e., lower percentage of DSB) than SpyCas9 but produce higher amounts of nicked template than SpyCas9.

FIG. 12 shows an alternative means of illustrating the differences in the cleavage (endonuclease and nickase) activity of ancestral Cas enzymes in comparison to SpyCas9. The linearization and nick rate are shown plotted against AnCas age for all of LFCA, LBCA and LSCA compared to SpyCas9. The higher nick rate of the SpyCas9 in absolute terms result of the fitting assuming an equal initial point for all the enzymes, which is true for t=0 minute but not for t>0 minute, because higher nickase activity measured by nicked cleavage results in a lower rate. As such, a negative value for the nick rate is shown. This is a conversion of the percentage of cleaved (nicked or linearized) plasmid template as shown in FIG. 11 into time units which provides a negative lambda parameter of the exponential decay shown in FIG. 10. LBCA and LSCA have a higher linearization rate than LFCA, but still lower than SpyCas9 and hence the trend is for linearization rate to decrease with ancestral age. In contrast, the nick rate increases with ancestral age.

FIGS. 13a-b illustrate PAM determination for LBCA and LSCA. FIG. 13a shows a PAM wheel from LBCA and LSCA PAM sequencing. LBCA and LSCA do not show specificity of recognition for any 3 nucleotide PAM. Similar results were obtained using 7 nucleotides. FIG. 13b shows results of an in vitro cleavage assay of plasmid DNA with different PAM sequences comparing LFCA, LBCA, LSCA and SpyCas9. LBCA (10 nM) nicked all the plasmids with different PAM within 10 minutes of reaction. LSCA showed similar preference but has higher linearization rate cleavage.

FIG. 14 shows results of testing the same ancestral Cas9 enzymes for endonuclease activity on single-stranded DNA. It is shown that the three ancestral enzymes cleave single-stranded DNA with or without gRNA. As expected, SpyCas9 was unable to cleave the same single stranded DNA.

FIGS. 15A-E show the activity of AnCas endonucleases on a supercoiled DNA substrate. FIG. 15A shows in vitro cleavage assay for SpCas9 and all AnCas on a 4007-bp substrate at different reaction times showing nicked and linear fractions. FIG. 15B shows the quantification of total cleavage at different reaction times and exponential fits (lines). FIG. 15C shows the quantification of nicked fraction for all AnCas and SpCas9 at different times. FIG. 15D shows the quantification of DSB cleavage. Single-exponential fits were used to obtain k_{cleave} and maximum fraction cleaved (amplitude). FIG. 15E shows the DSB fraction (left axis) and nicked fraction (right axis) plotted against evolutionary time.

FIGS. 16A-C show PAM determination of AnCas. FIG. 16A shows the PAM wheels (Krona plots) for all five AnCas and SpCas9, used as control. FIG. 16B shows the percentage of reads containing an NGG PAM sequence 3-4 bp downstream the cleavage position plotted against evolutionary time. FIG. 16C shows in vitro cleavage assay (DSB and nicked products) using a variety of PAM sequences represented by TNN and CCC as control. Incubation time was 10 minutes.

FIGS. 17A-G show sgRNA test and nuclease activity of AnCas on single-stranded substrates. FIG. 17A shows in vitro cleavage assay on a supercoiled DNA substrate of AnCas and SpCas9 using sgRNAs from different species. LFCA [FCA], LBCA [BCA] and SpCas9 are shown. FIG. 17B shows the quantification of in vitro cleavage for all AnCas and SpCas9 using the different sgRNAs. FIG. 17C shows in vitro cleavage assay on an 85-nt ssDNA fragment at different incubation times for LFCA [FCA], LBCA [BCA] and SpCas9. FIG. 17D shows in vitro cleavage assay on a 60-nt ssRNA at different incubation times for LFCA [FCA], LBCA [BCA] and SpCas9. FIG. 17E shows the quantification of fraction cleavage of ssDNA at different times and exponential fits for determination of kinetics parameters. In both FIG. 17C and FIG. 17D, the control lane is the same for the three proteins. FIG. 17F shows the quantification of fraction cleavage of ssRNA at different times and exponential fits for determination of kinetics parameters. All kinetics parameters are summarized in Table 2. FIG. 17G shows the results from an ELISA test of Anti-Cas9 rabbit antibody against SpCas9, LFCA [FCA], LBCA [BCA] and BSA, used as control.

FIG. 18 shows in vitro site-specific editing measure in HEK293T cells by NGS targeted sequencing using Illumina technology, in 3 independent targets.

FIGS. 19A-D show the activity of LFCA [FCA] H838A endonucleases on a supercoiled DNA substrate. FIG. 19A shows in vitro cleavage assay for LFCA [FCA] H838A on a 4007-bp substrate at different reaction times showing nicked and linear fractions. FIG. 19B shows the quantification of total cleavage fraction at different reaction times and exponential fits (lines). FIG. 19C shows the quantification of fraction nicked at different times. FIG. 19D shows the quantification of DSB cleavage. Single-exponential fits were used to obtain k_{cleave} and maximum fraction cleaved (amplitude).

FIG. 20 shows the posterior probability distribution for each inferred residue of all ancestral AnCas endonucleases. The residue with the highest posterior probability is assigned at each position. In all cases, posterior probability average is close to 1 except for LFCA [FCA] which shows an average value of 0.74.

FIGS. 21A-B show the activity of AnCas endonucleases at different temperature and pH values. FIG. 21A shows the quantification of total cleavage at different temperatures in the range 5-60° C. FIG. 21B shows the quantification of total cleavage at different pH in the range 4-9.5.

FIG. 22 shows PAM wheels (Krona plots) for all five AnCas and SpCas9 including 7-nucleotides PAM analysis. A preference for NGG PAM is observed except for LFCA [FCA].

FIG. 23 shows traffic light reporter cleavage assay. The relative NHEJ frequency is estimated by the number of RFP-positive cells and is normalized to SpCas9.

FIG. 24 shows a comparative assessment of PAM preferences for two AnCas [LFCA and LBCA] versus the wild-type Streptococcus pyogenes Cas9 [SpCas9], the so-called “ancestral Cas9 protein” of WO 2021/084533 A1 (SEQ ID NO: 268 of WO′533) [Anc. Cas], and the so-called “near-PAMless Cas9 proteins SpG and SpRY” of Walton et al. (2020. Science. 368(6488):290-296) [SpRY and SpG, respectively]. PAM preference for each of these nucleases is indicated, with N=any nucleotide and R=A or G.

### DETAILED DESCRIPTION

The starting sequence population for obtaining a functional ancestral Cas variant by the strategy now taught may preferably be, as exemplified, a population of Cas9 sequences from bacterial species in existence, whereby a phylogenetic tree can be constructed based on sequence alignment information. Computer-implemented methods for constructing such trees are well known in the field involving sequence alignment and recognition of conserved regions. However, as noted above, it is not excluded that the phylogenetic tree may be constructed of sequences of another class II Cas enzyme type.

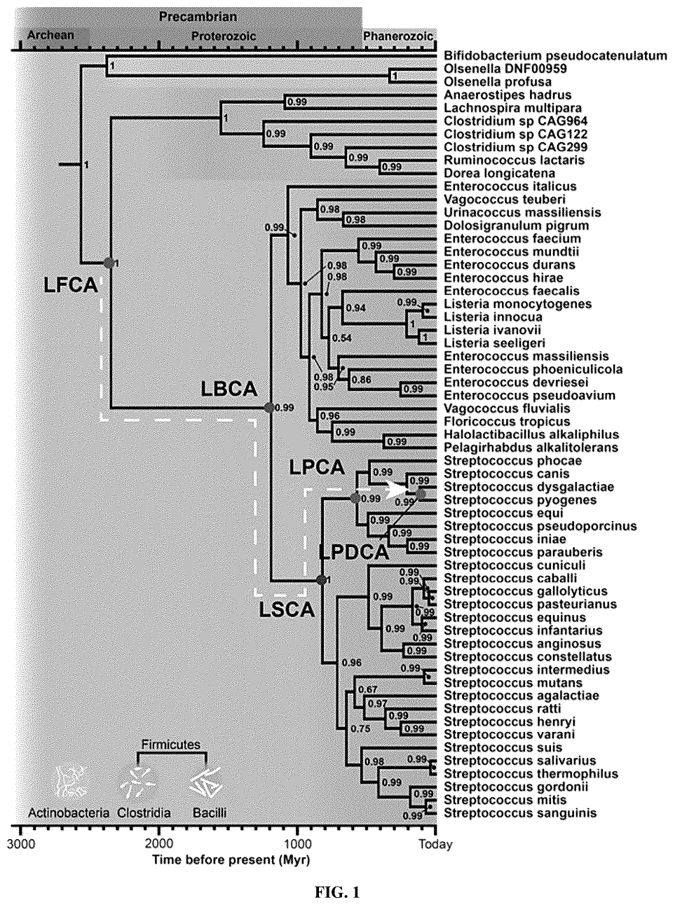

Preferably, the starting sequences will span more than one genus. For example, as illustrated in the Example section, in seeking an advantageous ancestral Cas9 variant, a plurality of Cas9 sequences may be selected from two or more of Streptococcus, Enterococcus, Listeria, Clostridium, Pelagirhabdus, Halolactibacillus, Floricoccus, Vagococcus, Urinacoccus, Vagococcus, Dorea, Ruminococcus, Lachnospira, Anaerostipes, Oisenella and Bifidobacterium. More than one species sequence may be chosen from each selected genus, e.g., 2, 3, 4 or more, e.g., up to 25 or more, e.g., 28, sequences from Streptococcus species. As noted above, LSCA is a common ancestor evolutionary traceable to all 28 Streptococci species listed in Table 1 including Streptococcus pyogenes, as shown by FIG. 1.

More preferably, the starting population of sequences will span more than one class of a phylum of interest. Thus, as indicated above, in seeking an advantageous ancestral Cas9 variant, it has been found useful to combine in the starting population Cas9 sequences derived from both Bacilli and Clostridia classes of bacteria. For example, the starting population of sequences may desirably comprise at least multiple sequences derived from different species of Streptococcus, multiple sequences derived from different species of Enterococcus, multiple sequences derived from different species of Listeria and multiple sequences derived from species of Clostridium. The diversity of the starting population may be further expanded to cross phyla as illustrated by inclusion of some Actinobacteria sequences in the starting population of Cas9 sequences employed in the Example section. Desirably, the starting population of Cas sequences may span a plurality of sub-types.

Starting from a compiled phylogenetic tree from known Cas sequences, evolutionary routes to predicted ancestral forms may be compiled, which may equate with many millions of years predating today. A selected ancestral variant sequence obtained in accordance with the invention may equate with an evolutionary period of at least 500 million years from the present, for example at least 700-800 million years, or even 1000 million years or more. As noted above, the evolutionary period may equate with as long as 2-3 Bys, e.g., about 2.2 to 2.4 Bys.

As shown by FIG. 1, LFCA Cas is such a reconstructed ancestor of Cas enzymes derived from evolutionary route analysis off a phylogenetic tree of Cas9 sequences derived from a population of existing bacterial species spanning Clostridia and Bacilli (both bacterial genera of the Firmicutes phylum) and supplemented with some Actinobacteria. It can be thought of as an ancestor of an earlier ancestral member of an evolutionary route to existing Cas9 enzymes of Bacilli origin (the reconstructed class ancestor designated LBCA Cas, with SEQ ID NO: 2) and an ancestor to a reconstructed ancestor of a broad variety of Cas enzymes of more specifically Streptococcus origin (the reconstructed genus ancestor LSCA Cas, with SEQ ID NO: 3). LSCA is in turn an ancestor to a reconstructed ancestor of a smaller selection (8 out of 28) of Cas enzymes of Streptococcus origin (the reconstructed ancestor designated LPCA Cas, with SEQ ID NO: 4) and LPCA is in turn an ancestor of Streptococcus pyogenes and Streptococcus dysgalactiae sequences (the reconstructed ancestor LPDCA Cas, with SEQ ID NO: 5).

As used herein and in the accompanying Figures, the terms “Bys”, “Bya” and “Gya” are used interchangeably to refer to billions of years.

As noted above, LFCA Cas is represented by node 63 of the illustrated evolutionary route and has the amino acid sequence shown in SEQ ID NO: 1. It shows high production level as well as high efficiency targeting and editing of DNA both in vitro and in human cells.

Thus, now provided as an aspect of the invention is a Cas nuclease comprising or consisting of the LFCA Cas having the amino acid sequence of SEQ ID NO: 1, representing a preferred example of a functional ancestral Cas obtained by adoption of the strategy taught herein for identification of such novel Cas enzymes. The LFCA Cas is deemed evolutionarily related to SpyCas9 but with a number of advantageous differences which render it an especially preferred AnCas nuclease.

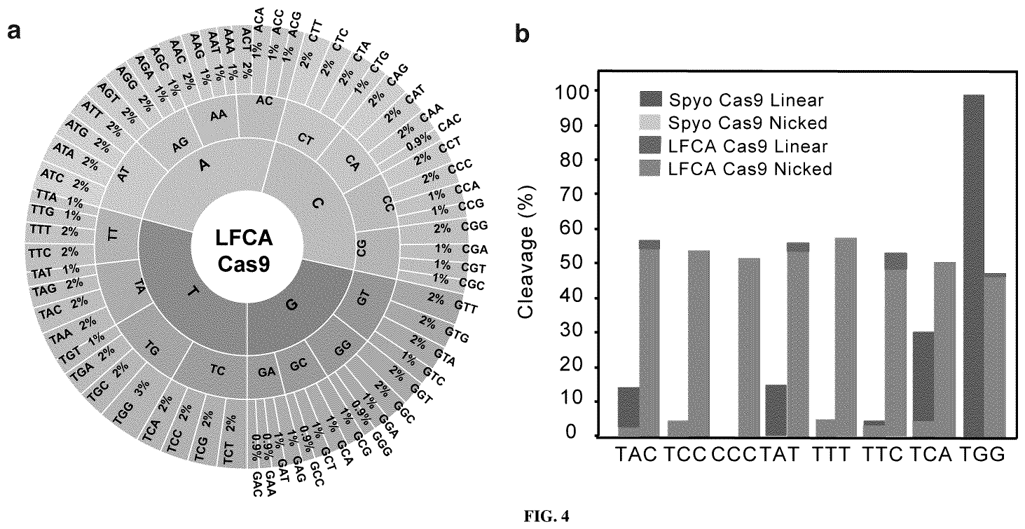

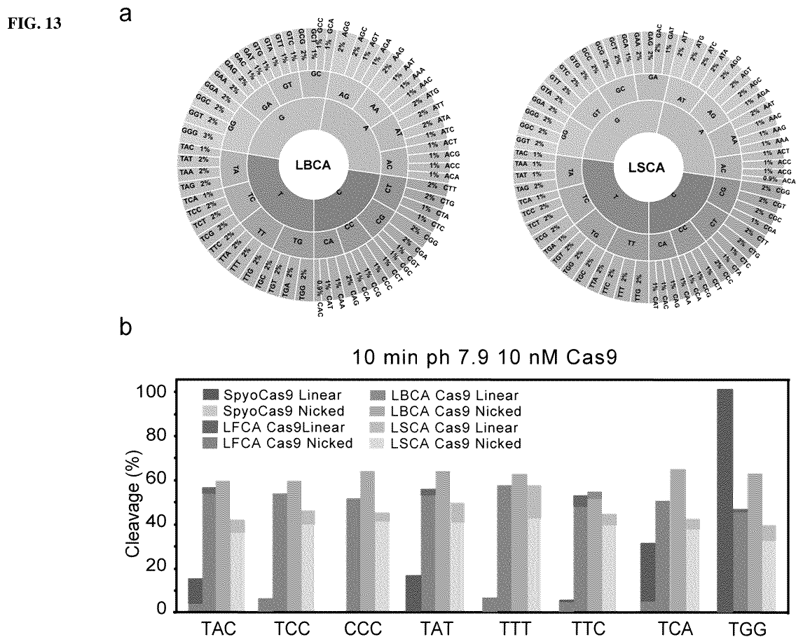

By way of interesting properties of LFCA Cas, it is now reported that:
(i) LFCA Cas is not known in nature and has only 54% of sequence identity with SpyCas9. Nevertheless, it can employ a sgRNA with the 3′ end of a SpyCas sgRNA for guide RNA/Cas protein interaction as shown in the Example section;
(ii) in contrast to SpyCas9, it exhibits time-separable nicking activity followed by endonuclease activity on double-stranded plasmid DNA providing a SpyCas9 PAM sequence and under conditions for endonuclease cleavage of the same plasmid by SpyCas9;
(iii) it exhibits broad PAM specificity; as shown by the PAM wheel of FIG. 4a, LFCA Cas did not show specificity of PAM recognition. The cleavage data of FIG. 4b show that it nicked plasmid DNA regardless of 3 nucleotide sequence provided as a proposed PAM sequence within 10 minutes at 10 nM. In contrast, SpyCas9 under the same conditions cut almost 100% of the plasmid containing its canonical PAM sequence, TGG, and showed low or no activity with other PAM sequences. Similar results were obtained using 7 nucleotide variant sequences for PAM provision. Thus, under the conditions tested, LFCA Cas can be designated “PAMless”;
(iv) it shows high flexibility in gRNA requirement; as shown by FIG. 8, it can utilize a sgRNA with a Cas9 tracrRNA component corresponding to that of any of a plurality of existing bacterial species and spanning a wide variety including Streptococcus thermophilus, Enterococcus faceium, Clostridium perfringens and Finegoldia magna, as well as Streptococcus pyogenes;
(v) LFCA Cas showed higher cleavage activity then SpyCas9 at low temperatures (from 4 to 20° C.) and at pH 7.9, which was observed as nicking activity;
(vi) at higher temperatures (from 53 to 60° C.), it exhibited higher thermal stability than SpyCas9 with both nickase and endonuclease activity being observed;
(vii) in pH stability tests at different pHs at 37° C., LFCA Cas maintained higher activity in acidic conditions than Spy Cas9. At alkaline pH, the activity of LFCA Cas remained the same, where SpyCas9 exhibited its optimal performance;
(viii) LFCA Cas shows cleavage activity for a single-stranded DNA substrate as shown in FIG. 13. As is well known, this is not an activity of SpyCas9 under normal usage conditions in the gene modification field.

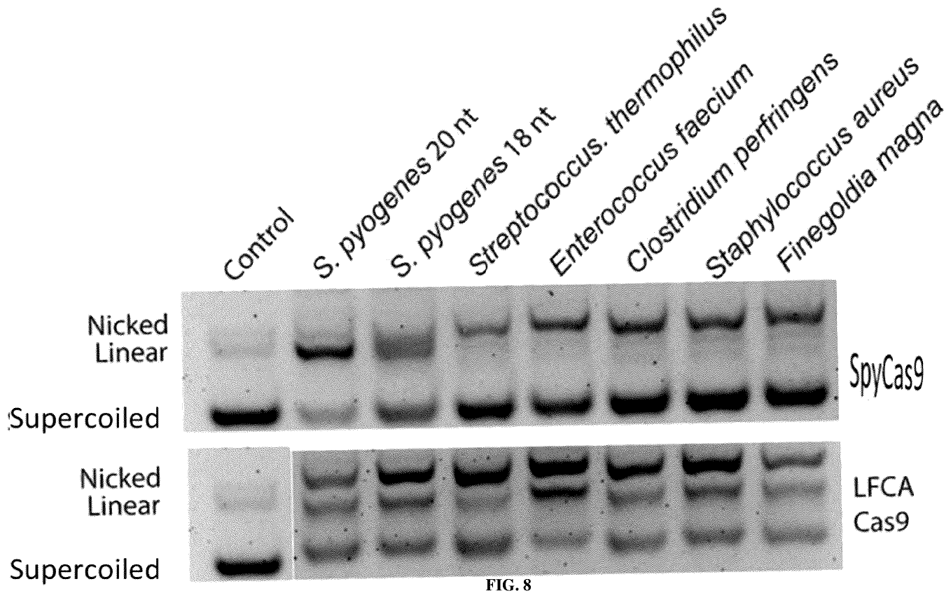

In human cells (exemplified herein with HEK293T cells), LFCA Cas has been shown to be capable of driving indel formation at a targeted locus (exemplified herein with the AAVS1 locus) when expressed in such cells with a suitable gRNA. Furthermore, ability to drive knock-in genetic modification at the same locus has been confirmed as shown in FIGS. 6 and 7.

Also provided as an aspect of the invention are Cas nucleases comprising or consisting of the LBCA Cas, LSCA Cas, LPCA Cas and LPCDA Cas having the amino acid sequence of SEQ ID NO: 2, 3, 4 and 5, respectively. Each of LBCA Cas, LSCA Cas, LPCA Cas and LPCDA Cas share interesting properties with LFCA Cas, which differentiate them as Cas enzymes from known Cas9 enzymes. Of particular interest, for example, is the higher percentage of nicked plasmid template produced compared to SpyCas as demonstrated additionally for LBCA Cas and LSCA Cas in the Example section (equating with a higher nick rate to plasmid linearization rate compared to SpyCas as demonstrated by exemplification below). As noted above, the percentage of nicked plasmid template (and/or nick rate) has interestingly been found to be increased in this group of enzymes with ancestral age, this feature being most pronounced in LFCA Cas. In contrast, the rate of linearization and/or percentage of double stranded breaks was found to decrease with ancestral age.

Thus, the present invention relates to a Cas nuclease comprising or consisting of:
(i) the Cas nuclease designated as LFCA Cas and having the amino acid sequence set forth in SEQ ID NO: 1;
(ii) the Cas nuclease designated as LBCA Cas and having the amino acid sequence set forth in SEQ ID NO: 2;
(iii) the Cas nuclease designated as LSCA Cas and having the amino acid sequence set forth in SEQ ID NO: 3;
(iv) the Cas nuclease designated as LPCA Cas and having the amino acid sequence set forth in SEQ ID NO: 4;
(v) the Cas nuclease designated as LPDCA Cas and having the amino acid sequence set forth in SEQ ID NO: 5; or
   a variant of such a Cas nuclease which retains one or more of the following distinguishing characteristics compared to SpyCas9:
(a) a higher percentage of nicked DNA plasmid template and/or a lower percentage of linearized DNA plasmid template under conditions whereby SpyCas9 results in substantially exclusively linearized DNA plasmid template;
(b) a higher nick rate and lower linearization rate on a DNA plasmid target under conditions whereby SpyCas9 results in substantially exclusively linearization or almost exclusively linearization (preferably, in a ratio of linearized DNA plasmid target to nicked DNA plasmid template of at least about 4:1) while the variant provides observable nicked target;
(c) a relaxed PAM requirement comparable to any of LFCA Cas, LBCA Cas and/or LSCA Cas;
(d) an ability to cleave single-stranded DNA;
(e) an ability to use a sgRNA in which the targeting sequence is linked to a tracrRNA component, wherein said tracrRNA component is selectable from the tracrRNA components of Cas9 gRNAs employed by a plurality of existing bacterial species.

As used herein, the term “variant” refers to a Cas nuclease having at least one amino acid mutation (e.g., addition, substitution or deletion) compared to the sequence of any one of SEQ ID NO: 1, SEQ ID NO: 2, SEQ ID NO: 3, SEQ ID NO: 4, and SEQ ID NO: 5, respectively. Typically, a Cas nuclease variant shares at least 60% of sequence identity, preferably at least 65%, 70%, 75%, 80%, 85%, 90%, 95%, 96%, 97%, 98%, 99% or more of sequence identity with the amino acid sequence of SEQ ID NO: 1, SEQ ID NO: 2, SEQ ID NO: 3, SEQ ID NO: 4, or SEQ ID NO: 5. It is to be understood that the amino acid sequence of a Cas variant is not 100% identical to any one of SEQ ID NO: 1, SEQ ID NO: 2, SEQ ID NO: 3, SEQ ID NO: 4, or SEQ ID NO: 5.

In some embodiments, the amino acid sequence of the Cas nuclease variant share at least 60%, 65%, 70%, 75%, 80%, 85%, 90%, 95% or more of sequence identity with the amino acid sequence of SEQ ID NO: 1.

In some embodiments, the amino acid sequence of the Cas nuclease variant share at least 75%, 80%, 85%, 90%, 95% or more of sequence identity with the amino acid sequence of SEQ ID NO: 2.

In some embodiments, the amino acid sequence of the Cas nuclease variant share at least 80%, 85%, 90%, 95% or more of sequence identity with the amino acid sequence of SEQ ID NO: 3.

In some embodiments, the amino acid sequence of the Cas nuclease variant share at least 85%, 90%, 95% or more of sequence identity with the amino acid sequence of SEQ ID NO: 4.

In some embodiments, the amino acid sequence of the Cas nuclease variant share at least 95%, 96%, 97%, 98%, 99% or more of sequence identity with the amino acid sequence of SEQ ID NO: 5.

In some embodiments, the Cas nuclease according to the invention has one or more amino acid changes by way of substitution or deletion, e.g., one or more conservative substitutions, whereby endonuclease and/or nickase activity is retained with the relaxed PAM specificity of LFCA Cas, LBCA Cas and/or LSCA Cas. In some embodiments, the Cas nuclease according to the invention has nickase activity. In some embodiments, the Cas nuclease according to the invention has relaxed PAM requirement. In some embodiments, the Cas nuclease according to the invention has no PAM requirement, i.e., the Cas nuclease is PAMless.

In a preferred embodiment, the Cas nuclease of the invention is LFCA with SEQ ID NO: 1 or a variant thereof, or is LBCA with SEQ ID NO: 2 or a variant thereof.

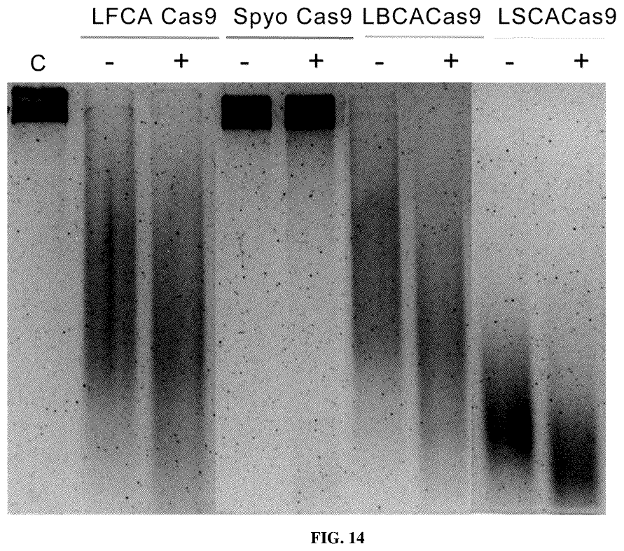

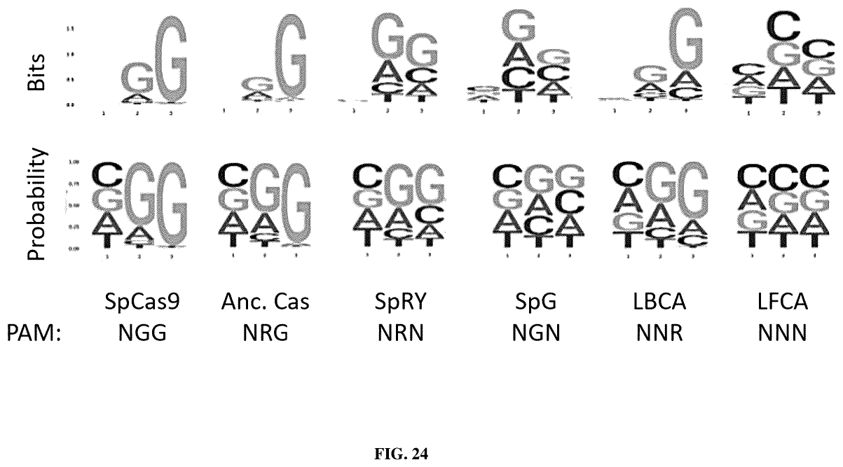

By way of example, LBCA Cas, LSCA Cas, LPCA Cas and LPCDA Cas have the following additional properties of interest:
(a) LBCA Cas is not known in nature and only has 70% identity to SpyCas9. LSCA Cas is not known in nature and only has 75% identity to SpyCas9. LPCA Cas is not known in nature and has 83.5% identity to SpyCas9. LPDCA Cas is not known in nature and has 97.5% identity to SpyCas9. Nevertheless, they can employ a sgRNA with the 3′ end of a SpyCas sgRNA for guide RNA/Cas protein interaction as shown in the Example section.
(b) In contrast to SpyCas9, as noted above, both of LBCA Cas and LSCA Cas have been found to share with LFCA Cas a relaxed PAM requirement (FIGS. 13a, 13b, and 24) and its ability to cleave single-stranded DNA (FIG. 14).

It will be recognized, however, that the novel AnCas enzymes exemplified herein are merely illustrative of the utility of the novel ancestral sequence reconstruction approach of the invention for attaining beneficial such enzymes for nucleic acid modification. It will be appreciated that the teaching herein opens up the attainment of other Cas9 variant enzymes, including variants of the exemplified AnCas enzymes, which share one or more of the same novel characteristics compared with SpyCas9, e.g.,
(a) a higher percentage of nicked DNA plasmid template and/or lower percentage of linearized DNA plasmid template (i.e., percentage of double stranded breaks) under conditions whereby SpyCas9 results in substantially exclusively linearized DNA plasmid template and/or
(b) a higher nick rate and lower linearization rate on a DNA plasmid target under conditions whereby SpyCas9 results in substantially exclusively linearization or almost exclusively linearization (as may equate with a ratio of linearized DNA plasmid target to nicked DNA plasmid template of at least about 4:1) while the variant provides observable nicked target and/or
(c) a relaxed PAM requirement comparable to any of LFCA Cas, LBCA Cas and LSCA Cas and/or
(d) an ability to cleave single-stranded DNA and/or single-stranded RNA, and/or
(e) an ability to use a sgRNA in which the targeting sequence is linked to a tracrRNA component, wherein said tracrRNA component is selectable from the tracrRNA components of Cas9 gRNAs employed by a plurality of existing bacterial species, e.g., including all of Streptococcus pyogenes, Streptococcus thermophilus, Enterococcus faceium, Clostridium perfringens and Finegoldia magna.

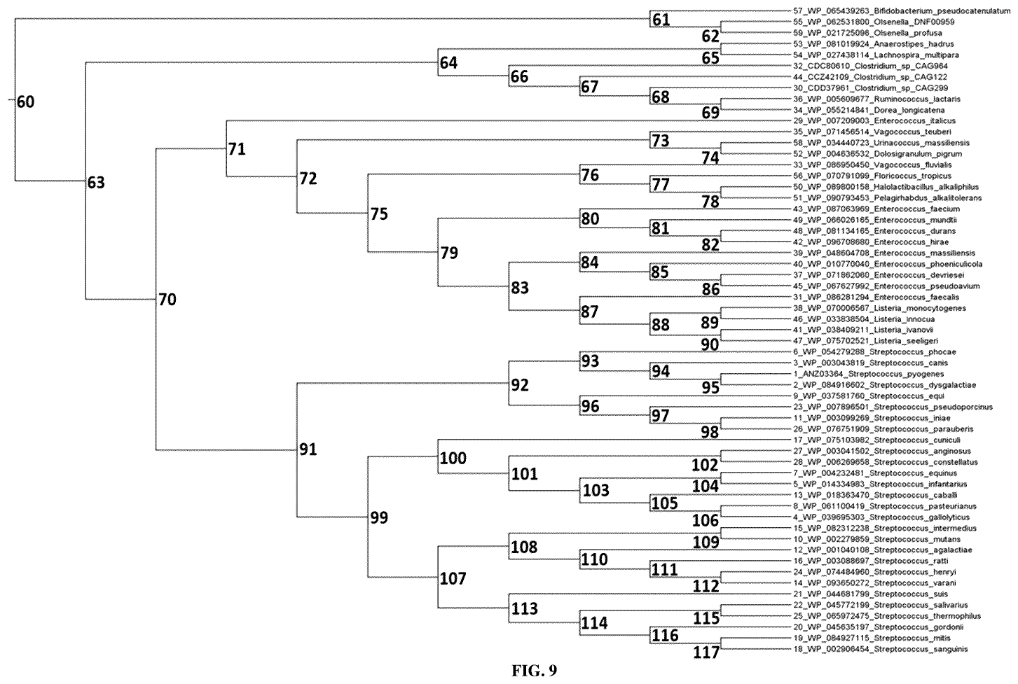

In particular, an aspect of the invention also provided herein are functional equivalents of the preferred ancestral Cas nucleases, which functional equivalents are represented by a node on the evolutionary route of FIG. 9. The amino acid sequences of these functional equivalents of Cas nucleases are given in the accompanying sequence listing, in SEQ ID NOs: 10 to 236.

the invention also extends to a method for obtaining an ancestral Cas nuclease, where the selected ancestral enzyme is evolutionarily traceable to existing Cas9 enzymes, preferably for example, evolutionary traceable to SpyCas9, and has one or more of characteristics (a) to (e) as noted above:
(a) a higher percentage of nicked DNA plasmid template and/or a lower percentage of linearized DNA plasmid template under conditions whereby SpyCas9 results in substantially exclusively linearized DNA plasmid template;
(b) a higher nick rate and lower linearization rate on a DNA plasmid target under conditions whereby SpyCas9 results in substantially exclusively linearization or almost exclusively linearization (preferably, in a ratio of linearized DNA plasmid target to nicked DNA plasmid template of at least about 4:1) while the variant provides observable nicked target;
(c) a relaxed PAM requirement comparable to any of LFCA Cas, LBCA Cas and/or LSCA Cas;
(d) an ability to cleave single-stranded DNA;
(e) an ability to use a sgRNA in which the targeting sequence is linked to a tracrRNA component, wherein said tracrRNA component is selectable from the tracrRNA components of Cas9 gRNAs employed by a plurality of existing bacterial species.

Especially preferred may be selection of an AnCas with the above-noted relaxed PAM specificity, possibly in combination with one or both of characteristics (a) and (d) or one or both of characteristics (a) and (e) or possibly in combination with all of (a), (d) and (e). As noted above, the selected AnCas may, for example, provide a ratio of linearized DNA plasmid target to nicked DNA plasmid template of between at least about 2.3:1 to at least 1:4 under conditions whereby SpyCas9 results in a ratio of linearized DNA plasmid target to nicked DNA plasmid template of at least about 4:1.

Thus, a method of the invention can be applied to obtain an ancestral Cas nuclease which has one or more of the following characteristics:
a ratio of linearized DNA plasmid target to nicked DNA plasmid template of between at least about 2.3:1 to at least 1:4 under conditions whereby SpyCas9 results in a ratio of linearized DNA plasmid target to nicked DNA plasmid template of at least about 4:1;
relaxed PAM requirement comparable to any of LFCA Cas, LBCA Cas and LSCA Cas;
ability to cleave single-stranded DNA and/or single-stranded RNA; and
ability to use a sgRNA in which the targeting sequence is linked to a tracrRNA component, wherein said tracrRNA component is selectable from the tracrRNA components of Cas9 gRNAs employed by a plurality of existing bacterial species

Such a method may further comprise converting such an AnCas nuclease to a variant which is either a nickase only or a deadCas with no nuclease activity and/or provide linkage to a non-nuclease effector, e.g., in a fusion protein. Such variants which are either a nickase only or a deadCas with no nuclease activity and/or a fusion protein are also contemplated as product per se in the present invention.

In some embodiments, the Cas nuclease is a non-nuclease modified deadCas variant of a nuclease according to the invention, which has been converted to a deadCas with no nuclease activity by catalytic site mutagenesis. In some embodiments, the Cas nuclease is catalytically dead. In some embodiments, the Cas nuclease is a deadCas.

In some embodiments, the Cas nuclease or Cas nuclease variant is linked with a non-nuclease effector for genetic modification or regulation. In some embodiments, the non-nuclease effector is a fusion protein comprising the Cas nuclease or Cas nuclease variant, and said non-nuclease effector.

In some embodiments, the ancestral Cas nuclease further has one or more of the following characteristics:
ability to cleave single-stranded DNA, and/or
ability to cleave single-stranded RNA.

In one embodiment, the ancestral Cas nuclease has relaxed PAM requirement comparable to any of LFCA Cas, LBCA Cas and LSCA Cas. In one embodiment, the ancestral Cas nuclease has no PAM requirement.

In some embodiments, the ancestral Cas nuclease has one or more of the following characteristics:
ability to cleave single-stranded DNA,
ability to cleave single-stranded RNA, and/or
relaxed PAM requirement comparable to any of LFCA Cas, LBCA Cas and LSCA Cas.

In some embodiments, the ancestral Cas nuclease has one or more of the following characteristics:
ability to cleave single-stranded DNA,
ability to cleave single-stranded RNA, and/or
no PAM requirement (i.e., PAMless activity) comparable to LFCA Cas.

Also provided are variants of the exemplified AnCas nucleases noted above which retain one or more of the distinguishing characteristics (a) to (e) above compared to SpyCas9. Again, especially preferred may be retention of the relaxed PAM specificity as exhibited by, for example, LFCA Cas, possibly in conjunction with one, two or all characteristics specified in (a), (b), (d) and (e) above, e.g., production of higher amounts of nicked template and/or lower amounts of linearized template (amount of double stranded breaks) compared with SpyCas9 as noted above and/or higher ratio of nick rate to linearization rate compared with SpyCas9 as noted above and/or ability to cleave single-stranded DNA. Preferably, all these characteristics will be retained.

As indicated above, especially preferred is LFCA Cas and variants thereof which are functionally equivalent, i.e., maintain all the characteristics of LFCA Cas (i) to (viii) listed above. LFCA Cas variants which retain at least relaxed PAM specificity and/or flexible tracrRNA usage as discussed above are, however, deemed highly favourable additions to the Cas enzyme toolbox.

In accordance with convention, the terms “linear activity”, and “endonuclease activity” are used synonymously herein to refer to nuclease activity for cleaving both strands of a double stranded DNA where it is provided in the form of plasmid. The terms “linearization activity rate”, “linearization rate” and “linear activity rate” are used herein synonymously to refer to a measure of the amount of target dsDNA that has been cleaved through both strands as a function of time. As used herein, “nickase” refers to a nuclease which cleaves only one strand of a dsDNA molecule, such as a plasmid, thereby generating a nick. “Nickase activity rate” and “nick rate” are used interchangeably and refer to a measure of the amount of target dsDNA that has been cleaved through one strand as a function of time. The nickase and/or linear activity rate may be tested by a method involving
(i) incubating Cas nuclease at 30 nM with gRNA at 1:1 ratio in a cleavage buffer (e.g., 100 mM NaCl, 50 mM Tris-HCl, 10 mM MgCl₂, 100 μg BSA, pH 7.9) at 37° C. for at least 5 minutes;
(ii) adding target DNA, e.g., a plasmid;
(iii) incubating, e.g., for 30 minutes;
(iv) stopping the cleavage reaction; and
(v) visualizing the final reaction products, e.g., by running the final reaction products on an agarose gel.

Using a method similar to the method described using an incubation time of 30 minutes, the preferred Cas nucleases of the invention may produce a ratio of linearized DNA plasmid target to nicked DNA plasmid template of between at least 2.3:1 to at least 1:4 under conditions whereby SpyCas9 results in a ratio of linearized DNA plasmid target to nicked DNA plasmid template of at least 4:1. Thus as noted above after 30 minutes, AnCas enzymes obtained in accordance with the invention such as LFCA, LBCA and LSCA may nick at least 30% of the DNA template up to at least 70%, e.g., about 80%, of the DNA template whereas under the same conditions SpyCas9 nicks about 10% of the DNA template in the same amount of time as illustrated in FIG. 11.

The percentage of DNA template with double stranded breaks (DSB) (i.e., linearized template) formed by the preferred Cas nucleases of the invention may be from 10% up to about 70%. The percentage of DNA template with double stranded breaks (DSB) formed by the preferred Cas nucleases of the invention may be at most 70%, 60%, 50%, 40%, 30%, 20%, or 10%. The percentage of DNA template with double stranded breaks (DSB) formed by the preferred Cas nucleases of the invention may be from 15% up to about 65%. The percentage of double stranded breaks (DSB) formed in a DNA template by the preferred Cas nucleases of the invention may be from 19% up to about 62%.

The percentage of DNA template with double stranded breaks (DSB) formed by LFCA Cas may be about 19%. The percentage of DNA template with double stranded breaks (DSB) formed by LBCA Cas may be around 36%. The percentage of DNA template with double stranded breaks (DSB) formed by LSCA Cas may be around 62%. Conversely, under the same experimental conditions as those used to test any one of the preferred Cas nucleases of the invention, the percentage of DNA template with double stranded breaks (DSB) formed by SpyCas9 is at least 70%, 75% or 80%.

The percentage of DNA template with nicks formed (i.e., nicked template produced) by the preferred Cas nucleases of the invention may be from 20% up to about 100%. The percentage of DNA template with nicks formed by the preferred Cas nucleases of the invention may be at least 30%, 40%, 50%, 60%, 70%, 80%, or 90%. The percentage of DNA template with nicks formed by the preferred Cas nucleases of the invention may be from 20% up to about 90%. The percentage of DNA template with nicks formed by the preferred Cas nucleases of the invention may be from 35% up to about 85%.

The percentage of DNA template with nicks formed by LFCA Cas may be around 80%. The percentage of DNA template with nicks formed by LBCA Cas may be around 65%. The percentage of DNA template with nicks formed by LSCA Cas may be around 35%. Conversely, under the same experimental conditions as those used to test any one of the preferred Cas nucleases of the invention the percentage of DNA template with nicks formed by SpyCas9 is at most about 20% or 10%.

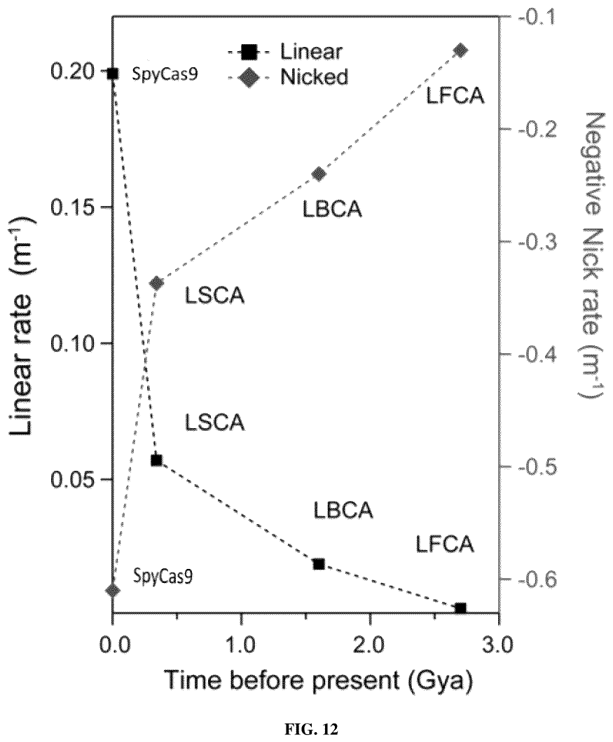

As shown in FIG. 12, the difference in cleavage activity of the preferred ancestral Cas enzymes of the invention can be illustrated by way of a linearization rate and a nick rate. Thus, it can be considered that the linearization rate of the preferred Cas nucleases of the invention may be from about 0.001 to about 0.1 m⁻¹. The nick rate of the preferred Cas nucleases of the invention may be from about −0.4 to about −0.1 m⁻¹. For example, the linearization rate and nick rate may be as shown in Table 2.

In some embodiments, the Cas nuclease variant is modified by catalytic site mutagenesis to retain just nickase activity. In some embodiments, the Cas nuclease variant is a Cas nickase. In some embodiments, the amino acid sequence of the Cas nuclease comprises one or more amino acid changes by way of substitution or deletion, e.g., one or more conservative substitutions, whereby endonuclease and/or nickase activity is retained with the relaxed PAM specificity of LFCA Cas.

From the above, it will be evident that LFCA Cas, LBCA Cas, LSCA Cas, LPCA Cas and LPCDA Cas and variants thereof as discussed above (and functional equivalents thereof, e.g., those represented by a node on the evolutionary route of FIG. 9 or others obtained in accordance with the ancestral reconstruction strategy of the invention) are seen as highly useful novel additions to the toolbox of Cas proteins, especially LFCA Cas with the highest observed nick rate. They may be used directly under conditions to promote nickase only action (although modification by catalytic site mutagenesis to retain just nickase activity as, for example, LFCA nickase, LBCA nickase and LSCA nickase, LPCA nickase and LPDCA nickase is not excluded). Any of LFCA Cas, LBCA Cas, LSCA Cas, LPCA Cas and LPCDA Cas or such variants may be linked, e.g., fused, with an effector protein for gene modification, e.g., a base editor such as a deaminase for base editing or a reverse transcriptase for prime-editing.

It will be appreciated that variants of any of LFCA Cas, LBCA Cas, LSCA Cas, LPCA and LPCDA Cas (or a corresponding nickase obtained by catalytic site mutagenesis) which have one or more amino acid changes by way of substitution or deletion, e.g., one or more conservative substitutions, may be similarly employed as a Cas9 endonuclease or Cas9 nickase provided endonuclease and/or nickase activity is retained. Of especial interest and forming part of the invention are such variants that also retain the relaxed PAM specificity as shown for LFCA Cas, LBCA Cas and/or LSCA Cas.

Variants of an AnCas nuclease obtained by the ancestral reconstruction strategy of the invention, or variants of a corresponding nickase obtained by catalytic site mutagenesis, which form part of the invention may, for example, have various degrees of sequence identity to the parent enzyme subject to retaining the desired distinguishing characteristic or characteristics. They may for example, have at least 80%, at least 90%, at least 95%, at least 96%, at least 97%, at least 98% or at least 99% sequence identity.

Like naturally-occurring Cas9 nucleases, any of LFCA Cas, LBCA Cas, LSCA Cas, LPCA Cas and LPCDA Cas or variants thereof as noted above (and functional equivalents thereof, e.g., those represented by a node on the evolutionary route of FIG. 9 or others obtained in accordance with the ancestral reconstruction strategy of the invention) may also be converted to a dCas with no nuclease activity by catalytic site mutagenesis and may further be linked, e.g., fused, with a non-nuclease effector protein.

Thus, LFCA Cas, LBCA Cas, LSCA Cas, LPCA Cas and LPCDA Cas, variants thereof as discussed above (and functional equivalents thereof, e.g., those represented by a node on the evolutionary route of FIG. 9 or others obtained in accordance with the ancestral reconstruction strategy of the invention) may be employed in the whole panoply of genetic modification techniques envisaged for naturally-occurring Cas9 nucleases of existing species and modified versions thereof. These extend to use in combination with an effector for gene modification or regulation such as a base editor where linkage is via an RNA extension of a guide and an RNA-binding domain as taught in WO 2017/011721 (Rutgers University, licensed to Horizon Discovery). See also Collantes et al., 2021. CRISPR J. 4(1):58-68).

Preferably, for example, the Cas enzyme variant linked or fused with a non-nuclease effector may be any AnCas obtained in accordance with the invention or variant thereof with nuclease activity which has been modified by conventional catalytic site mutagenesis to have nickase only activity or no nuclease activity, i.e., is a dCas, and exhibits the relaxed PAM specificity observed with, for example LFCA Cas, LBCA Cas and/or LSCA Cas.

The invention additionally provides nucleic acids for expression of the AnCas proteins described herein, including variants and functional equivalents thereof, e.g., expression vectors for expression of such proteins. Such vectors may be employed with a guide RNA, or guide RNA expressed from DNA. Thus, a combination of vectors providing an AnCas nuclease as herein taught or variant or functional equivalent thereof, and a suitable guide RNA, may be provided for transfection into cells. Such combinations including one or more vectors, including a vector for expression of, e.g., LFCA Cas, LBCA Cas, LSCA Cas, LPCA Cas or LPCDA Cas or a variant or functional equivalent thereof, in human cells, may be provided in the form of a pharmaceutical composition with a pharmaceutically acceptable excipient.

Preferably the Cas protein may be LFCA Cas or a corresponding nickase. It will be appreciated that the corresponding nickase of any of the ancestral enzymes herein taught may be so provided, e.g., an LBCA nickase, LSCA nickase, LPCA nickase or LPCDA nickase.

Thus, the present invention further relates to a nucleic acid capable of expressing a Cas nuclease or Cas nuclease variant according to the invention.

In some embodiments, the nucleic acid is a DNA or an RNA molecule. In some embodiments, the nucleic acid is a DNA molecule, e.g., a complementary DNA molecule. In some embodiments, the nucleic acid is an RNA molecule, e.g., a messenger RNA molecule.

In some embodiments, the nucleic acid is single stranded or double stranded. In some embodiments, the nucleic acid is single stranded. In some embodiments, the nucleic acid is double stranded.

In some embodiments, the nucleic acid comprises natural nucleotides. In some embodiments, the nucleic acid comprises a combination of natural and non-natural nucleotides.

In some embodiments, the nucleic acid is comprised in a vector. Non limitative examples of suitable vectors comprise plasmid, fosmid, cosmid, artificial chromosome or viral vector. In some embodiments, the vector is comprised in a nanoparticle, e.g., a lipid nanoparticle.

The present invention further relates to a combination of a vector comprising the nucleic acid according to the invention as described hereinabove, and a guide RNA for targeting the Cas nuclease or variant or functional equivalent thereof to a target DNA sequence, or a vector capable of expressing the guide RNA.

Alternatively, a novel AnCas nuclease as herein taught, such as LFCA Cas, LBCA Cas, LSCA Cas, LPCA Cas or LPCDA Cas or a variant or functional equivalent thereof, e.g., a corresponding nickase as discussed above, may be provided as a ribonucleoprotein (RNP) complex with a guide RNA for transfection into cells, e.g., by electroporation into isolated cells. Thus, the present invention further refers to a ribonucleoprotein complex comprising a Cas nuclease or Cas nuclease variant according to the invention, or a Cas nuclease or Cas nuclease variant and a guide RNA for targeting the Cas nuclease or Cas nuclease variant to a target DNA sequence.

It will be appreciated that the term “guide RNA” as used herein may be a single molecule targeting RNA (sgRNA) or, if suitable as for a naturally-occurring Cas9, a dual sequence RNA comprising (i) a DNA targeting segment comprising a nucleotide sequence complementary to the target sequence (the crRNA) and (ii) a protein-binding segment that interacts with the Cas protein (the tracrRNA).

In a further aspect, the invention provides a method for modifying or regulating a target nucleic acid sequence, e.g., a target DNA sequence, the method comprising contacting the target sequence with a complex comprising (i) a Cas protein as taught, e.g., LFCA Cas, LBCA Cas, LSCA Cas, LPCA Cas or LPCDA Cas, or a variant or functional equivalent thereof as discussed above, and (ii) a guide RNA for targeting the Cas protein to the target sequence, wherein either:
(a) said contacting is in vitro on an isolated target nucleic acid sequence or in a cell ex vivo, preferably with the proviso that methods of modifying the germ line identity of a human being are excluded; and/or
(b) the method is not a method of medical treatment practiced on the human or animal body.

The complex may further comprise a nucleic acid molecule encoding a transgene of interest, e.g., for introduction of this transgene of interest into the target DNA sequence.

Preferably, the Cas protein may be, for example, LFCA Cas, LBCA Cas, or another exemplified AnCas nuclease as noted above, which retains the same relaxed PAM requirement. Preferably, the Cas protein may be such an AnCas but modified to present only a nickase activity or no nuclease activity in the form of a fusion protein.

Such methods extend for example to genetic modification of human or animal cells ex vivo. Cas proteins as now taught, e.g., LFCA Cas, LBCA Cas and other AnCas, including variants and functional equivalents thereof, may also find use in relation to genetic modification in plants, e.g., by modifying target sequences, possibly but not exclusively, in protoplasts.

It will be appreciated that the invention also extends to a combination for use in therapeutic treatment by modifying or regulating a target nucleic acid sequence, for example a DNA sequence, wherein the combination comprises:
(i) a Cas protein as taught herein, e.g., LFCA Cas, LBCA Cas, LSCA Cas, LPCA Cas, or LPCDA Cas, or a variant or functional equivalent thereof as discussed above, or a polynucleotide capable of expressing the same, and
(ii) a guide RNA for targeting the Cas protein to a target nucleic acid sequence or a polynucleotide capable of expressing the same.

In particular, therapeutic treatment may include the prevention and/or treatment of genetic diseases. The combination may then further comprise a nucleic acid molecule encoding a transgene of interest, wherein said transgene of interest may, e.g., compensates a gene defect responsible for the genetic disease.

As indicated above, the low sequence identity of, for example LFCA Cas to SpyCas9, is considered advantageous in relation to contemplating such use. Such use may embrace for example Cas action in pathogenic bacteria or for manipulation of the gut microbiome or skin microbiome.

The following exemplification illustrates the invention with reference to both obtaining and testing of the Cas enzymes LFCA Cas, LBCA Cas and LSCA Cas, but as noted above it is envisaged that other ancestral Cas proteins with advantageous properties may be obtained by the same strategy depending on the choice of starting population of Cas enzyme sequences providing the phylogenetic tree for the evolutionary analysis. The predicted resurrection may be as old as 3 Bys.

### EXAMPLES

### Example 1

Ancestral Sequence Reconstruction of Cas Nucleases from Existing Cas9 Sequences

Sequences were collected of the gene cas9 from the Uniprot database from several Firmicutes bacterial species using as query the sequence SpyCas9 (Uniprot code: Q99ZW2). The search confirmed the existence of hundreds of sequences of cas9 genes from the phylum Firmicutes within the classes Bacilli and Clostridia. Some sequences from Actinobacteria were also found. After downloading 59 sequences (Table 1), a sequence alignment was constructed that confirmed the common origin of the Cas9 sequences with a portion of the sequences showing significant conservation. Using Bayesian inference (BEAST software), a phylogenetic tree was compiled to confirm the phylogenetic relationship of the sequences. Using maximum likelihood, five AnCas sequences were reconstructed to the Last Common Ancestor of Firmicutes (LFCA), going back ˜2.4 Bya (see FIG. 1). The evolutionary path from LFCA Cas to modern Streptococcus pyogenes was followed. LFCA Cas was found to only have around 50% identity with SpyCas9 (over 500 mutations). Other evolutionary routes were constructed such as those leading to Clostridium and Enterococcus species. Genes encoding ancestral Cas enzymes were synthesized, cloned into expression vectors, expressed in E. coli and purified in the lab. Five AnCas enzymes from the route LFCA to S. pyogenes (shown in bold in FIG. 1) were obtained. All 5 were found to be expressed at high levels, folded, and soluble despite sequence identities ranging from about 50 to nearly 95% with SpyCas9.

TABLE 1
Cas9 sequences utilized for ancestral reconstruction of ancient Cas9 variants

| Phylum | Class | Genus | Specie | Sequence |
|---|---|---|---|---|
| Firmicutes | Bacilli | Streptococcus | Streptococcus | WP_084916602.1 |
| | | | dysgalactiae | |
| Firmicutes | Bacilli | Streptococcus | Streptococcus canis | WP_003043819.1 |
| Firmicutes | Bacilli | Streptococcus | Streptococcus | WP_012962174.1 |
| | | | gallolyticus | |
| Firmicutes | Bacilli | Streptococcus | Streptococcus | WP_014334983.1 |
| | | | infantarius | |
| Firmicutes | Bacilli | Streptococcus | Streptococcus | WP_054279288.1 |
| | | | phocae | |
| Firmicutes | Bacilli | Streptococcus | Streptococcus | WP_157339387.1 |
| | | | equinus | |
| Firmicutes | Bacilli | Streptococcus | Streptococcus | WP_061100419 |
| | | | pasteurianus | |
| Firmicutes | Bacilli | Streptococcus | Streptococcus equi | WP_037581760.1 |
| Firmicutes | Bacilli | Streptococcus | Streptococcus | WP_002279859.1 |
| | | | mutans | |
| Firmicutes | Bacilli | Streptococcus | Streptococcus iniae | WP_003099269.1 |
| Firmicutes | Bacilli | Streptococcus | Streptococcus | AFV72233.1 |
| | | | agalactiae | |
| Firmicutes | Bacilli | Streptococcus | Streptococcus | WP_018363470.1 |
| | | | caballi | |
| Firmicutes | Bacilli | Streptococcus | Streptococcus | WP_003041502 |
| | | | anginosus | |
| Firmicutes | Bacilli | Streptococcus | Streptococcus | WP_093650272 |
| | | | varani | |
| Firmicutes | Bacilli | Streptococcus | Streptococcus | WP_082312238.1 |
| | | | intermedius | |
| Firmicutes | Bacilli | Streptococcus | Streptococcus ratti | WP_003088697.1 |
| Firmicutes | Bacilli | Streptococcus | Streptococcus | WP_002906454.1 |
| | | | sanguinis | |
| Firmicutes | Bacilli | Streptococcus | Streptococcus mitis | WP_084927115.1 |
| Firmicutes | Bacilli | Streptococcus | Streptococcus | WP_045635197.1 |
| | | | gordonii | |
| Firmicutes | Bacilli | Streptococcus | Streptococcus suis | WP_044681799.1 |
| Firmicutes | Bacilli | Streptococcus | Streptococcus | WP_002891502.1 |
| | | | salivarius | |
| Firmicutes | Bacilli | Streptococcus | Streptococcus | WP_007896501.1 |
| | | | pseudoporcinus | |
| Firmicutes | Bacilli | Streptococcus | Streptococcus | WP_074484960.1 |
| | | | henryi | |
| Firmicutes | Bacilli | Streptococcus | Streptococcus | WP_065972475.1 |
| | | | thermophilus | |
| Firmicutes | Bacilli | Streptococcus | Streptococcus | WP_076751909.1 |
| | | | parauberis | |
| Firmicutes | Bacilli | Streptococcus | Streptococcus | WP_006269658.1 |
| | | | constellatus | |
| Firmicutes | Bacilli | Streptococcus | Streptococcus | WP_032464890.1 |
| | | | pyogenes | |
| Firmicutes | Bacilli | Streptococcus | Streptococcus | WP_075103982.1 |
| | | | cuniculi | |
| Firmicutes | Bacilli | Enterococcus | Enterococcus | WP_007209003.1 |
| | | | italicus | |
| Firmicutes | Bacilli | Enterococcus | Enterococcus | WP_048604708.1 |
| | | | massiliensis | |
| Firmicutes | Bacilli | Enterococcus | Enterococcus | OJG69383.1 |
| | | | phoeniculicola | |
| Firmicutes | Bacilli | Enterococcus | Enterococcus | NTL99667.1 |
| | | | faecalis | |
| Firmicutes | Bacilli | Enterococcus | Enterococcus | WP_071862060.1 |
| | | | devriesei | |
| Firmicutes | Bacilli | Enterococcus | Enterococcus hirae | WP_096708680.1 |
| Firmicutes | Bacilli | Enterococcus | Enterococcus | WP_087063969.1 |
| | | | faecium | |
| Firmicutes | Bacilli | Enterococcus | Enterococcus | WP_067627992.1 |
| | | | pseudoavium | |
| Firmicutes | Bacilli | Enterococcus | Enterococcus | WP_081134165.1 |
| | | | durans | |
| Firmicutes | Bacilli | Enterococcus | Enterococcus | WP_023519017.1 |
| | | | mundtii | |
| Firmicutes | Bacilli | Vagococcus | Vagococcus fluvialis | WP_086950450.1 |
| Firmicutes | Bacilli | Vagococcus | Vagococcus teuberi | WP_071456514.1 |
| Firmicutes | Bacilli | Listeria | Listeria | WP_003739838.1 |
| | | | monocytogenes | |
| Firmicutes | Bacilli | Listeria | Listeria ivanovii | WP_038409211.1 |
| Firmicutes | Bacilli | Listeria | Listeria innocua | WP_010991369.1 |
| Firmicutes | Bacilli | Listeria | Listeria seeligeri | WP_075702521.1 |
| Firmicutes | Bacilli | Halolactibacillus | Halolactibacillus | WP_089800158.1 |
| | | | alkaliphilus | |
| Firmicutes | Bacilli | Pelagirhabdus | Pelagirhabdus | WP_090793453.1 |
| | | | alkalitolerans | |
| Firmicutes | Bacilli | Dolosigranulum | Dolosigranulum | WP_004636532.1 |
| | | | pigrum | |
| Firmicutes | Bacilli | Anaerostipes | Anaerostipes hadrus | WP_055196588.1 |
| Firmicutes | Bacilli | Floricoccus | Floricoccus tropicus | WP_070791099.1 |
| Firmicutes | Bacilli | Urinacoccus | Urinacoccus | WP_034440723 |
| | | | massiliensis | |
| Firmicutes | Clostridia | Clostridium | Clostridium sp | CDD37961.1 |
| | | | CAG299 | |
| Firmicutes | Clostridia | Clostridium | Clostridium sp | CDC80610.1 |
| | | | CAG964 | |
| Firmicutes | Clostridia | Clostridium | Clostridium sp | CCZ42109.1 |
| | | | CAG122 | |
| Firmicutes | Clostridia | Lachnospira | Lachnospira | WP_027438114.1 |
| | | | multipara | |
| Firmicutes | Clostridia | Ruminococcus | Ruminococcus | WP_005609677.1 |
| | | | lactaris | |
| Firmicutes | Clostridia | Dorea | Dorea longicatena | WP_055214841.1 |
| Actinobacteria | Coriobacteriaceae | Olsonella | Olsenella_DNF00959 | WP_062531800.1 |
| Actinobacteria | Coriobacteriaceae | Olsonella | Olsenella profusa | WP_021725096.1 |
| Actinobacteria | Bifidobacteriaceae | Bifidobacterium | Bifidobacterium | WP_065439263.1 |
| | | | pseudocatenulatum | |

See the material and methods section below for further information on the evolutionary analysis.

### Testing the Endonuclease Activity of AnCas Enzymes

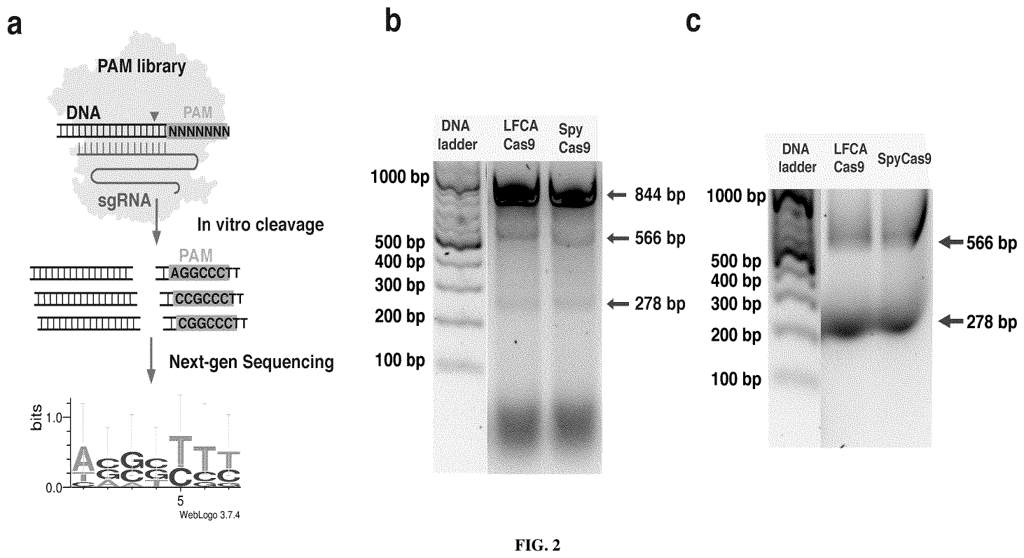

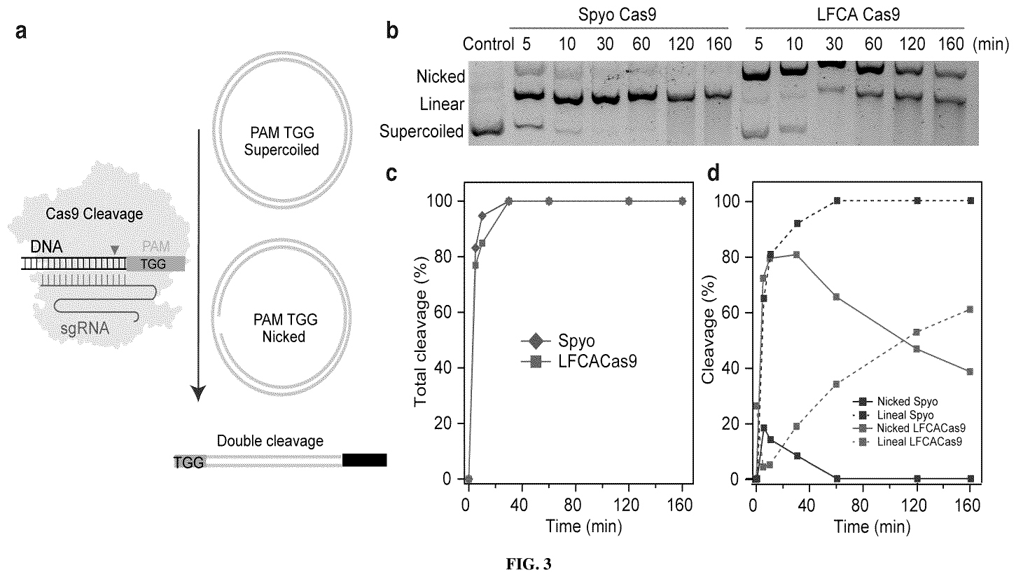

To test if the ancestral Cas showed endonuclease activity, a DNA library with 7 random nucleotides (NNNNNNN) was designed and a gRNA to target the sequence after these nucleotides. This random DNA library was needed as the LFCA Cas PAM sequence was unknown (FIG. 2a). The sequences for provision of the random library were cloned in the plasmid pUC18 and a sequence of 844 bp was amplified to produce a linear DNA fragment. SpyCas9 cleavage produces two fragments, one with 566 bp and a smaller one with 278 bp that contains the PAM sequence. Both LFCA Cas and SpyCas9 incubation with gRNA targeting the PCR fragment successfully produced both fragments (FIG. 2b) confirming that the ancestral Cas has catalytic activity. In addition, a DNA fragment containing the S. pyogenes PAM (NGG) extracted from the library by PCR was recognized by both Cas enzymes. LFCA was able to recognize S. pyogenes PAM and cleave the DNA (FIG. 2c).

To study the DNA cleavage dynamics of LFCA Cas, a DNA fragment containing the S. pyogenes PAM (TGG) was cloned and incubated with LFCA Cas or SpyCas9 at different times ranging from 5 to 160 minutes. Both enzymes were incubated with gRNA and target DNA and the reaction stopped by adding loading buffer and EDTA. The samples were run on a 1%-agarose gel to detect supercoiled, nicked and linear DNA (FIG. 3a). On the agarose gel (FIG. 3b), the different DNA conformations after Cas enzyme activity were observed. The band intensity was measured and the total cleavage by both enzymes at different times was calculated (FIG. 3c). Both enzymes cleaved almost 100% of the plasmid DNA after 10 minutes. However, it was observed that LFCA Cas cuts one strand of the DNA and the other strand cleavage increases with time, i.e., nicking and endonuclease activity were separated in time as shown in FIG. 3d. In comparison SpyCas9 cuts the two strands of DNA simultaneously.

In another line of experiments, the AnCas genes were synthesized and cloned into pBAD/gIII expression vectors, carrying an arabinose inducible promoter and a gIII-encoding signal that directs the AnCas to the periplasmic space. All AnCas were expressed at high levels in Escherichia coli BL21 cells.

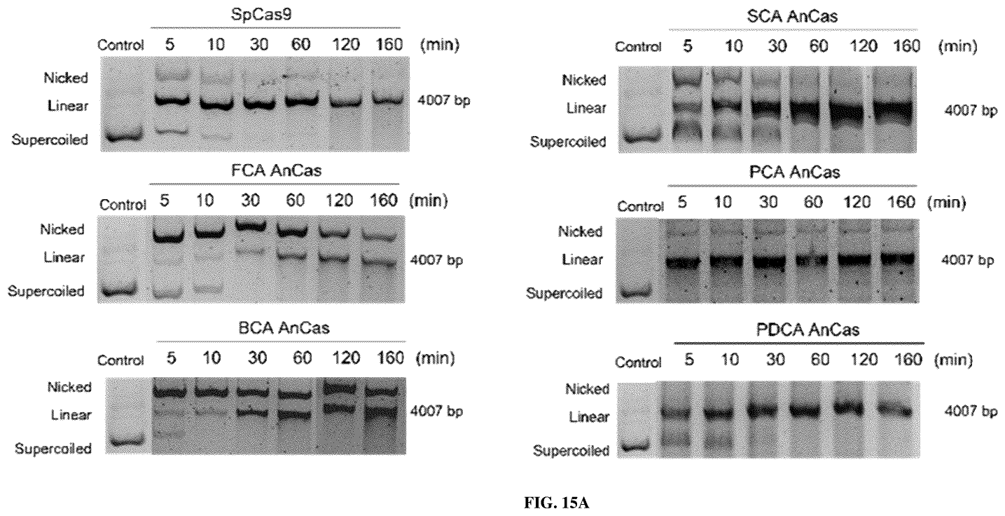

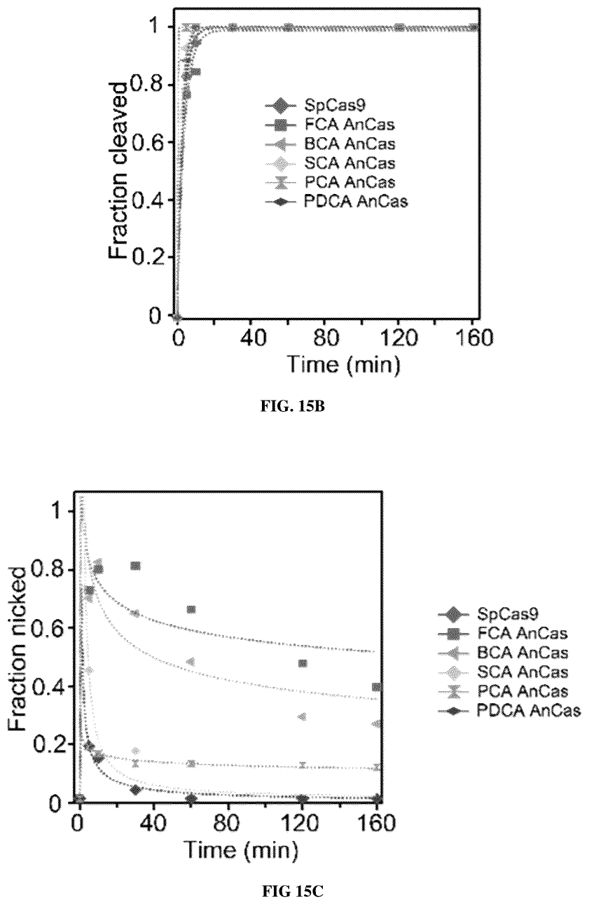

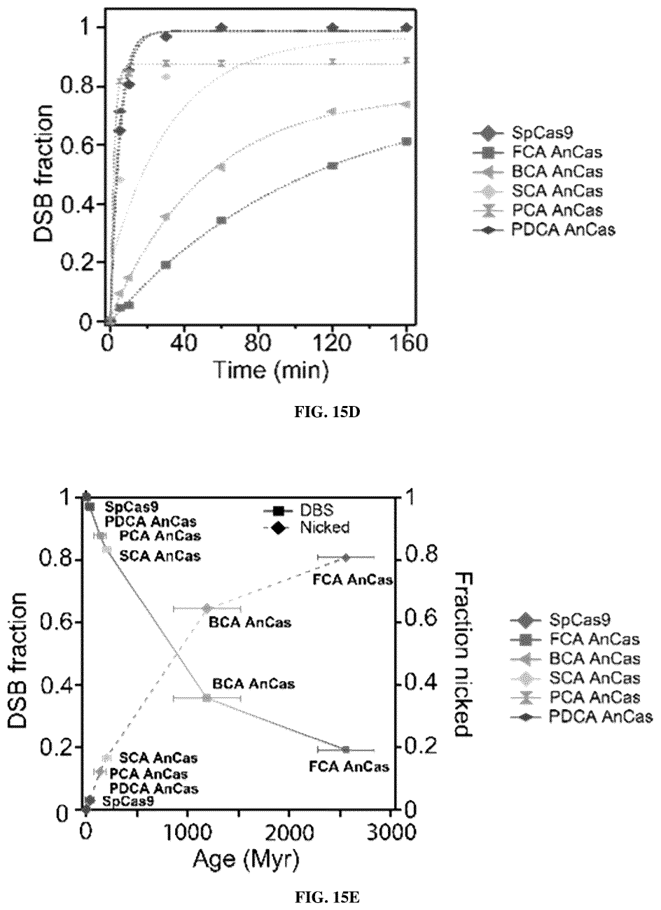

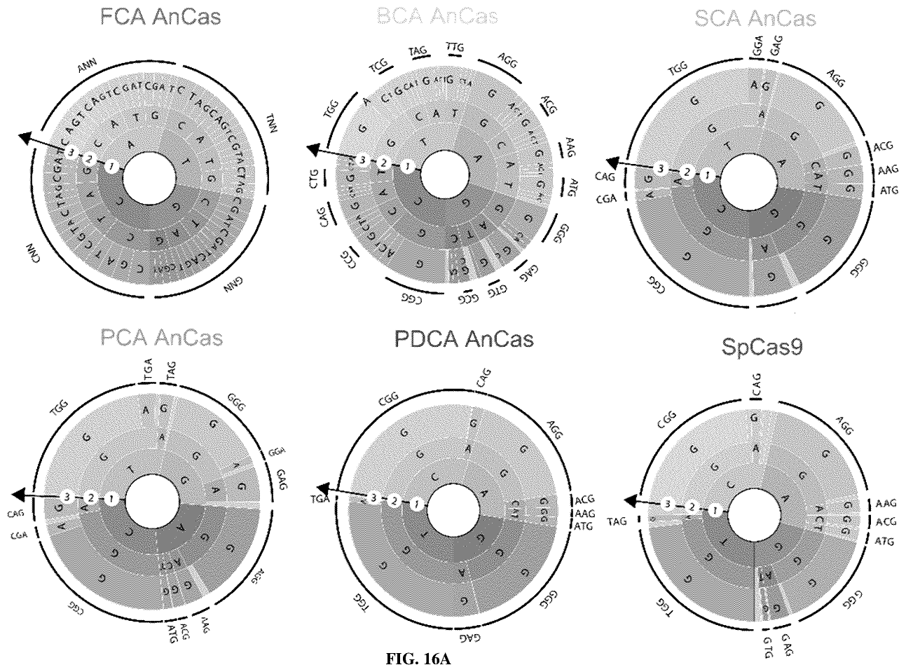

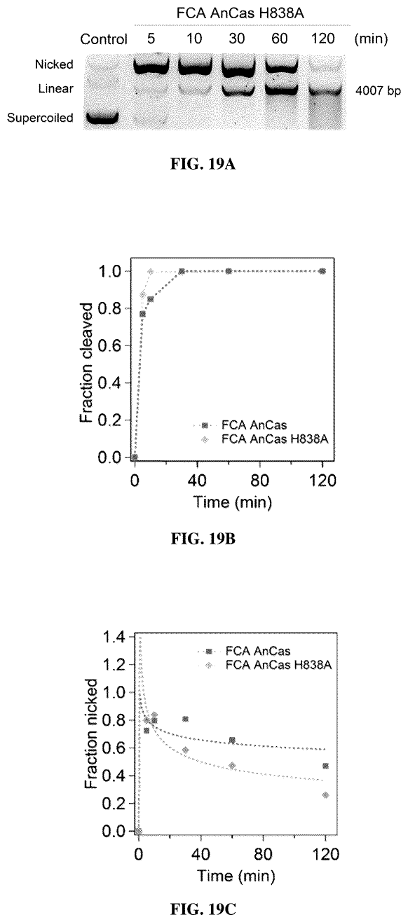

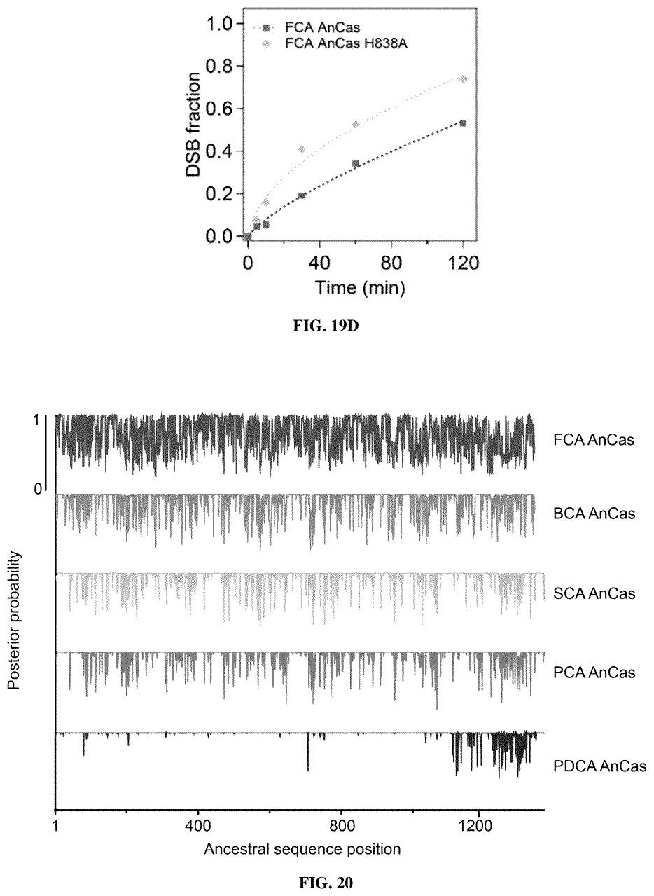

The activity test was started by assuming a simplistic scenario by which AnCas would recognize a sgRNA from S. pyogenes as well as its canonical 5′-NGG-3′ PAM sequence. An sgRNA containing a 20 nt-long spacer region targeted towards a DNA fragment upstream of a TGG PAM was designed, all placed in a 4007 bp-supercoiled plasmid. In vitro cleavage assays were carried out by incubating AnCas or SpCas9 with target DNA together with the sgRNA at different digestion times. Although with clear differences in cleavage efficiency, all enzymes tested produced both relaxed and linear products, indicative of nickase and DSB activity, respectively. As expected, SpCas9 showed nicked product after short incubation times and linear products after longer incubation times (FIG. 15A). However, in the case of AnCas, the behavior changed from the oldest LFCA AnCas to the more recent enzymes (FIG. 15A). LFCA AnCas mostly showed nickase activity and the DSB activity becomes prominent only at times over 60 minutes. The other AnCas showed a progressive behavior with more intensive DSB activity in the younger AnCas (FIG. 15A). Both the nicked and linear fraction for each AnCas and SpCas9 were quantified and plotted versus incubation time in three forms, total cleavage (FIG. 15B), nicked fraction (FIG. 15A) and linear fraction (FIG. 15D), demonstrating the progressive decrease of nicked fraction and increase of linear fraction. SpCas9 had the higher proportion of linear products, the oldest LFCA AnCas had the highest proportion of nicked fraction. The percentage of linear and nicked products were plotted versus geological time demonstrating an evolutionary trend from nickase to DSB activity (FIG. 15E). The structural differences observed in LFCA AnCas protein related to HNH domain displacement, together with the evolutionary trend from nickase to DSB activity, suggests that the oldest AnCas, LFCA AnCas, could display an ancestral HNH domain with a reduced or suppressed activity. To examine this, the in vitro activity of a H838A LFCA AnCas mutant was tested (H840A with respect to the wild-type SpCas9 amino acid sequence). The mutant was able to produce nicked and, surprisingly, linear products, showing a profile practically identical to that obtained with wild type LFCA AnCas (FIG. 19A-D). These results suggest that LFCA AnCas may contain an immature HNH domain, with the RuvC domain responsible for the nickase and DSB activity observed in LFCA AnCas, as has been previously shown in some type V effector nucleases that lack the HNH domain, such as Cpf1 (Cas12a), Cas14 (Cas12f) or CasΦ (Cas12j)27-30.

TABLE 2
Cas9 nucleases linearization (linear) and nick (nickase) rates:

| | | Negative | |
|---|---|---|---|
| Time before | Linear | Nick rate | Cas |
| present (GYA) | rate (m⁻¹) | (m⁻¹) | Nuclease |
| 0 | 0.199 | −0.61 | SpyCas9 |
| 0.34 | 0.057 | −0.337 | LSCA |
| 1.6 | 0.019 | −0.24 | LBCA |
| 2.7 | 0.0027 | −0.13 | LFCA |

In another line of experiments, the DNA cleavage activity of two AnCas, LBCA Cas and LSCA Cas, was compared with the DNA cleavage activity of LFCA Cas and SpyCas9.

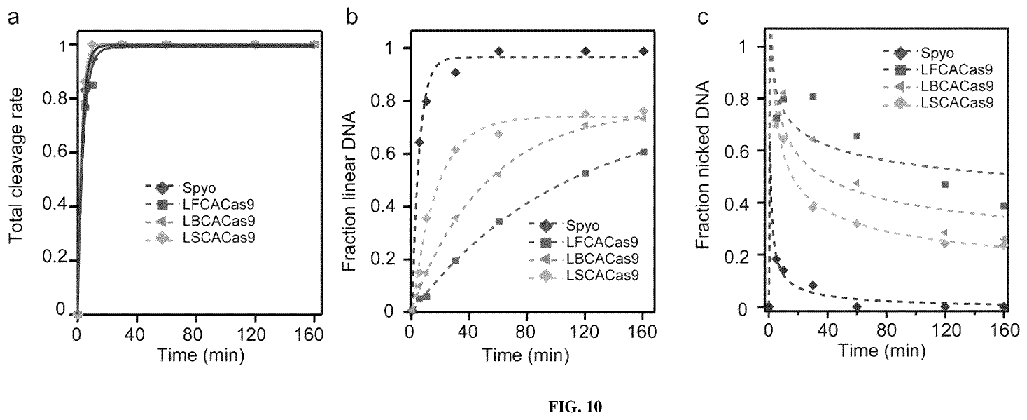

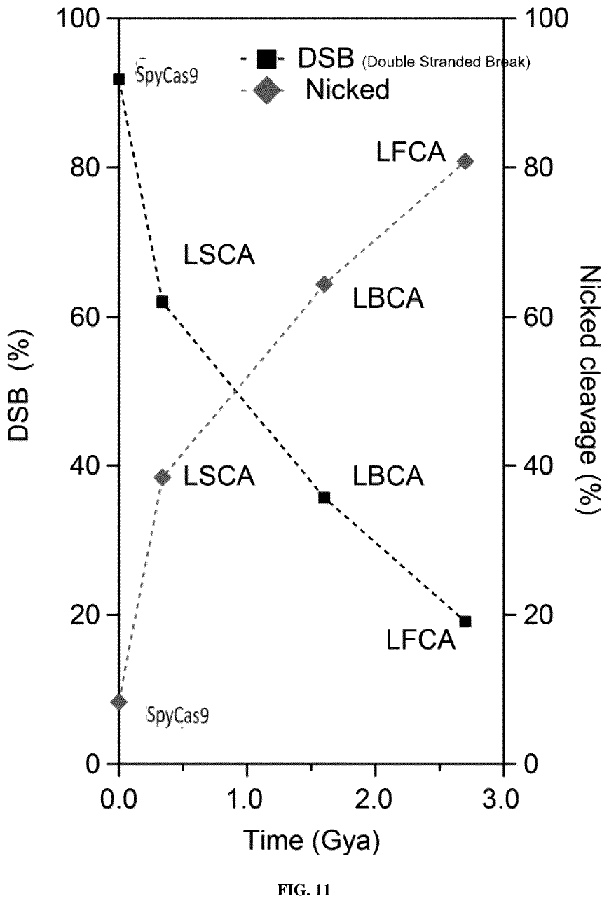

A plasmid containing the TGG PAM sequence after the target sequence was incubated with each enzyme for different times. The cleavage rates of the four enzymes were similar (FIG. 10a) when the total cleavage was compared. The linearization and nick rates were however different depending on the enzyme. LBCA Cas and LSCA Cas were shown to have a higher linearization rate than LFCA Cas, but still lower than SpyCas9 (FIG. 10b). In contrast, the nick rates of LBCA Cas and LSCA Cas were lower than that of LFCA Cas, but higher than the nick rate of SpyCas9 (FIG. 10c).

The cleavage activity of the AnCas enzymes could be seen to follow a trend which is shown in FIG. 11. The percentage of double stranded breaks after an incubation time of 30 minutes can be seen to be highest for SpyCas9 and decreases with the age of ancestral Cas enzymes (i.e., the percentage of double stranded breaks can be seen to decrease with the older the ancestral enzyme as follows: % DSB for SpyCas9>% DSB for LSCA Cas>% DSB for LBCA Cas>% DSB for LFCA Cas).

The opposite correlation was seen for percentage of nicked template formed (i.e., % nicked cleavage for SpyCas9<% nicked cleavage for LSCA Cas<% nicked cleavage for LBCA Cas<% nicked cleavage for LFCA Cas).

By converting the percentages of DSBs and nicked template shown in FIG. 11 to a time-dependent value, a linearization rate and nick rate could be calculated and plotted. The linearization and nick rates seem to follow a trend which is seen when the rates are plotted versus the age of each ancestor (FIG. 12).

### PAM Determination

A DNA fragment amplified by PCR from a DNA library as described in the PAM library construction section below was used to determine the PAM specificity of LFCA Cas. The LFCA Cas with gRNA and the DNA library were incubated for 1 hour and the reaction products run in a 2%-agarose gel. The small fragment of 278 bp was extracted from the agarose gel and was analyzed by Ion Torrent Next Generation Sequencing (NGS). From the sequencing data, the frequency of each PAM recognized by LFCA Cas was analyzed and the total proportion against the overall frequency of each PAM in the library was calculated. The calculated frequencies were plotted in a PAM wheel to visualize the PAM affinity of the ancestral Cas (FIG. 4a). The wheel shows the loss of PAM preference of LFCA Cas compared with SpyCas9.

To confirm this result, an in vitro-PAM determination assay was carried out with a DNA plasmid carrying a target DNA with different PAM combinations (TNN 3 nucleotide combinations and CCC). LFCA and SpyCas9 (10 nM) incubated for 10 minutes with each PAM had different cleavage activities. LFCA Cas showed similar nicking activity with all the PAMs tested. In contrast, SpyCas9 showed cleavage with the TGG PAM (its well-known canonical PAM sequence), and showed lower cleavage than LFCA Cas with the other PAMs (FIG. 4b).

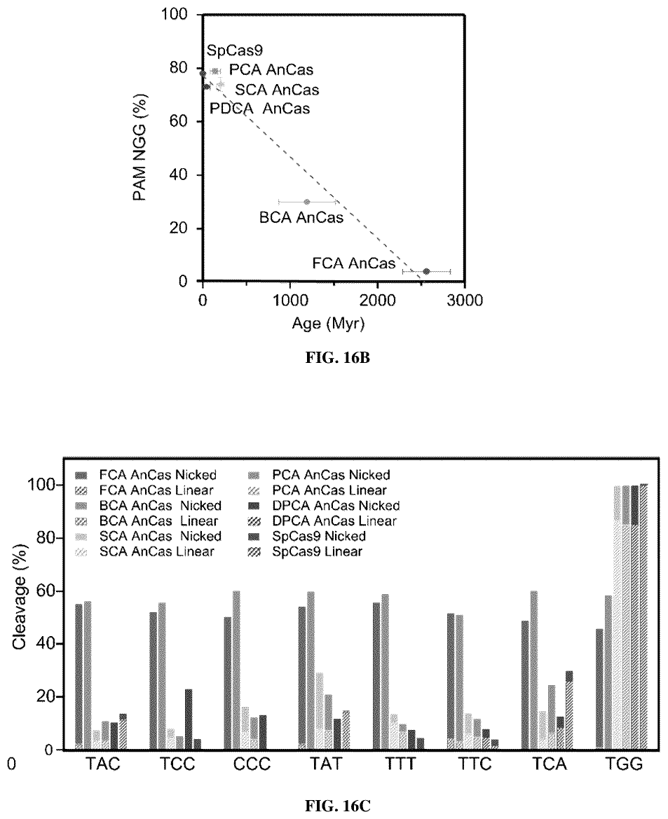

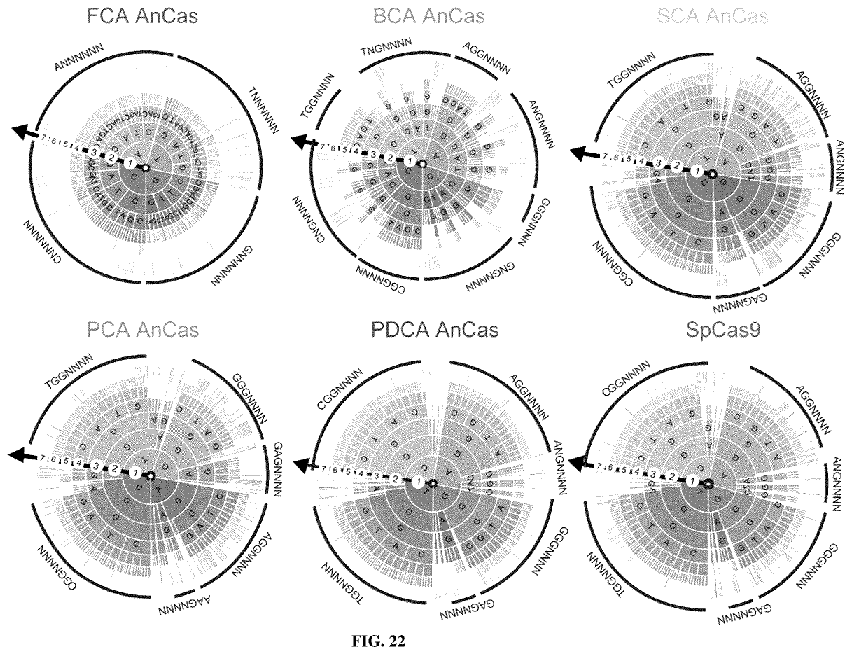

In another line of experiments, the ability of the AnCas endonucleases to recognize different PAMs was investigated. To determine the preferred PAM sequence of each AnCas, a DNA library containing a target sequence followed by seven random nucleotides (NNNNNNN) that corresponded to all possible PAMs was designed. An sgRNA was designed using the scaffold of S. pyogenes and 20 nucleotides complementary to the target sequence. PCR primers were designed to amplify an 844 bp-fragment containing both the target and PAM sequence, which was used as a substrate for AnCas and SpCas9. In vitro digestion using the purified Cas protein, and the transcribed sgRNA, was performed with the PCR target. All five AnCas produced two fragments, one of 566 bp and a smaller one of 278 bp that would contain the PAM sequences recognized by the AnCas. The small fragment was purified, sequenced by Next-generation Sequencing (NGS) and analyzed to determine PAM sequence diversity from each AnCas, enabling to infer how evolution changed it. FIG. 16A summarizes the results of PCR cleavage assay in the form of PAM wheels (Krona plot) for the five AnCas and SpCas9. As previously observed, LFCA AnCas exhibited no preference for any PAM sequence tested. For the other Cas proteins, a preference for specific nucleotides in the target-proximal positions 2 and 3 was detected (FIG. 22). For instance, in the case of LBCA AnCas, a slight preference for NGG was revealed, although additional PAM sequences were also detected (NNG). For more recent AnCas, the NGG bias was more pronounced (data not shown).

After analyzing all the sequences, the percentage of reads with an NGG PAM were plotted versus the geological time of each AnCas, as estimated in the phylogenetic analysis. A trend that reflects an NGG enrichment over time was observed, demonstrating that NGG fidelity is an evolutionary trait that portrays a gradual progression from PAMless to NGG preference in more recent Streptococci ancestors (FIG. 16B). This confirms the hypothesis of an evolving adaptive response for PAM recognition, which would be expected as the number of spacers acquired by the host cell increases over time. Eventually, a strong PAM recognition ability would be required to avoid self-cleavage of the CRISPR locus, especially, in a scenario in which the increment of DSB activity (deleterious in most prokaryotes) over nickase activity might further increase the evolutionary pressure on this ability. Although PAM-permissive (or “near-PAMless”) Cas9 variants have been previously described, LFCA AnCas is the first fully PAMless Cas9 endonuclease ever reported to our knowledge.

To further probe the PAMless ability of LFCA AnCas, an in vitro-PAM determination assay was designed to test cleavage of a target DNAs adjacent to a total of six PAM sequences (TAC, TCC, TAT, TTT, TTC and TAC) within the general TNN PAM. A CCC PAM was also included in the set to verify possibilities other than an initial T nucleotide. AnCas effectors together with the sgRNA were incubated with each of the target DNAs for 10 minutes and cleavage products were verified by agarose gel (data not shown). Both nicked and linear products were observed, demonstrating the cleavage activity with all TNN PAM sequences. In the case of SpCas9, only TGG PAM demonstrated double-stranded break of the supercoiled DNA substrate. The percentage of nicked and linear products was quantified for each PAM, represented in FIG. 16C. For the oldest AnCas (LFCA Cas and LBCA Cas), the percentage of cleavage was similar for all PAM sequences tested, with mostly nicked products, as expected given the incubation time. In the case of younger AnCas and SpCas9, the cleavage fraction reached high levels for TGG PAM, corroborating the NGG PAM preference. In the case of CCC control, the cleavage profile was similar to those obtained from non-NGG PAM sequences.

In another line of experiments, PAM determination was carried out using DNA fragments amplified by PCR from a DNA library again as described in the PAM library construction section below. LBCA Cas or LSCA Cas were incubated with gRNA and the DNA library for 1 hour with run of the reaction in a 2%-agarose gel. A small fragment of 278 bp was extracted from the agarose gel and analyzed by Ion Torrent Next Generation Sequencing (NGS). From the sequencing data the frequency of each PAM recognized by the AnCas enzyme was determined and the total proportion against the overall frequency of each PAM in the library was calculated. The calculated frequencies were plotted in a PAM wheel to visualize the PAM affinity for both AnCas (FIG. 13a). The wheel shows similar PAM preference of LBCA and LSCA Cas as exhibited by LFCA Cas.

A PAM determination assay was performed in vitro with a DNA plasmid carrying a target DNA with different PAM nucleotide combinations (TNN). LBCA Cas and LSCA Cas (10 nM) were incubated for 10 mins with each PAM (FIG. 13b). LBCA Cas showed a similar cleavage rate or even higher with some PAMs than LFCA Cas. LSCA Cas also showed cleavage with all the PAM sequences tested but has lower activity. A higher linear activity throughout for all the PAMs tested was seen compared with LFCA Cas, highlighting the higher linear cleavage rate of LBCA Cas and LSCA Cas.

Finally, we wanted to compare the PAM preferences for LFCA Cas (which has no preference for any PAM sequence as demonstrated above) and for LBCA Cas (which was shown to be near-PAMless with a preference for NNG sequence), versus Cas9 proteins disclosed in the art, in particular:
a so-called “ancestral Cas9 protein” disclosed in WO 2021/084533 A1 (SEQ ID NO: 268 of WO′533); and
the near-PAMless Cas9 proteins “SpG”* and “SpRY” ** of Walton et al. (2020. Science. 368(6488):290-296).

### * SpG: D1135L/S1136W/G1218K/E1219Q/R1335Q/T1337R

### ** SpRY: A61R/L1111R/D1135L/S1136W/G1218K/E1219Q/N1317R/A1322R/R1333 P/R1335Q/T1337R

In all cases, a wild-type SpCas9 control was included to benchmark.

A DNA library containing seven random nucleotides was designed and cloned into pUC18 plasmids (Genscript). This random library was transformed in XL1blue Escherichia coli and amplified several times to achieve the maximal variability in the PAM sequences.

PAM determination assay was performed by incubating 3 nM of DNA library plasmid with 30 nM of each tested Cas protein in cleavage buffer, together with a gRNA targeting the 20 nucleotides upstream of the 7 random nucleotides. The reaction was incubated for 1 hour at 37° C. and stopped by adding 6× loading dye (NEB) with EDTA and run on 2% agarose gel. Gels were dyed with SYBR gold (ThermoFisher Scientific) and imaged on a ChemiDoc XRS+ System (Bio-Rad). PAM library-specific PCR-based amplification was performed using adapters and specific oligos:

| | SEQ ID NO | Sequence |
|---|---|---|
| P5_2 | 240 | AATGATACGGCGACC |
| | | ACCGAGATCTACAC |
| P705 | 241 | CAAGCAGAAGACGGC |
| | | ATACGAGATAGGAGT |
| | | CCGTGACTGGAGTCC |
| | | TCTCTATGGGCAGTC |
| | | GGTGA |
| P706 | 242 | CAAGCAGAAGACGGC |
| | | ATACGAGATCATGCC |
| | | TAGTGACTGGAGTCC |
| | | TCTCTATGGGCAGTC |
| | | GGTGA |
| P707 | 243 | CAAGCAGAAGACGGC |
| | | ATACGAGATGTAGAG |
| | | AGGTGACTGGAGTCC |
| | | TCTCTATGGGCAGTC |
| | | GGTGA |
| P708 | 244 | CAAGCAGAAGACGGC |
| | | ATACGAGATCCTCTC |
| | | TGGTGACTGGAGTCC |
| | | TCTCTATGGGCAGTC |
| | | GGTGA |
| Index1 | 245 | ATCACCGACTGCCCA |
| | | TAGAGAGGACTCCAG |
| | | TCAC |
| Read2 | 246 | GTGACTGGAGTCCTC |
| | | TCTATGGGCAGTCGG |
| | | TGAT |

The fragment was sequenced by Illumina sequencing and the reads were mapped to the reference sequence using Geneious Prime (2020 version). Illumina miSeq reads were aligned against amplified sequence with minimap2 for short reads to filter unspecific sequences. Then, reads with 3 nucleotides before the PAM region were selected from the aligned reads. The nucleotides in the region of interest were extracted using a custom script. Finally, logo plots of the PAM region were obtained with ggseqlogo and the PAM wheel of each sample was graphically represented with KronaTools.

Our data showed that, among the 6 different Cas proteins that were tested, only LFCA Cas was totally PAMless. LBCA Cas was the second Cas protein with the least restrictive PAM requirement. All other Cas proteins had more restrictive PAM preferences (FIG. 24).

Interestingly, the so-called “ancestral Cas9 protein” disclosed in WO 2021/084533 A1 displayed a NGG PAM requirement, identical to that of wild-type SpCas9.

These data therefore confirm that our AnCas proteins are truly PAMless or at least near-PAMless, contrary to the those disclosed in the art.

gRNA Recognition

The promiscuity for PAM recognition exhibited by the oldest AnCas raised the question of whether these AnCas would also show promiscuity towards gRNA recognition as well. The reconstruction of an ancestral gRNA would have been ideal; however, the variability in sequence of crRNA repeats and tracrRNA from different species makes this very difficult. To overcome this limitation and still evaluate the promiscuity of AnCas, modern sgRNAs from different species were tested. A total of five sgRNAs were selected from Streptococcus thermophilus, Enterococcus faecium, Clostridium perfingens, Staphylococcus aureus and Finegoldia magna, covering several Firmicutes classes. These sgRNAs were selected following previous studies on sgRNA classification and function, in which sgRNAs were divided into seven clusters. These distinct sgRNAs were contrasted against S. pyogenes guides containing spacers of two sizes, 18 and 20-nucleotide long, referred as “18 sgRNA” and “20 nt sgRNA”, respectively.

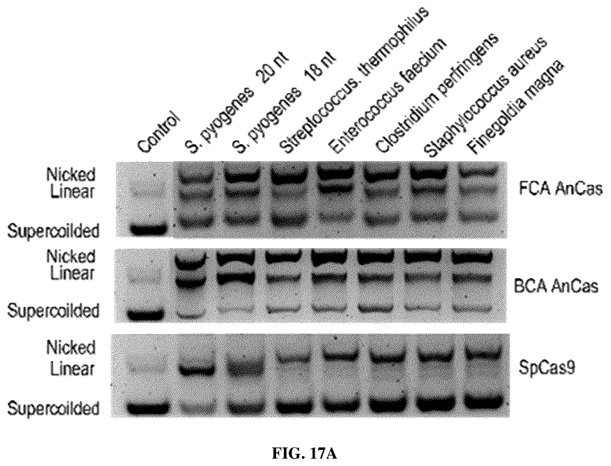

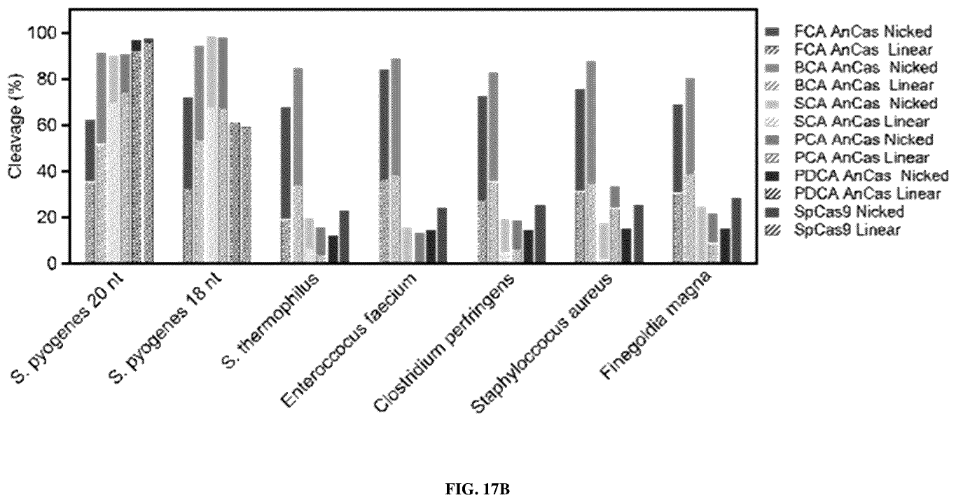

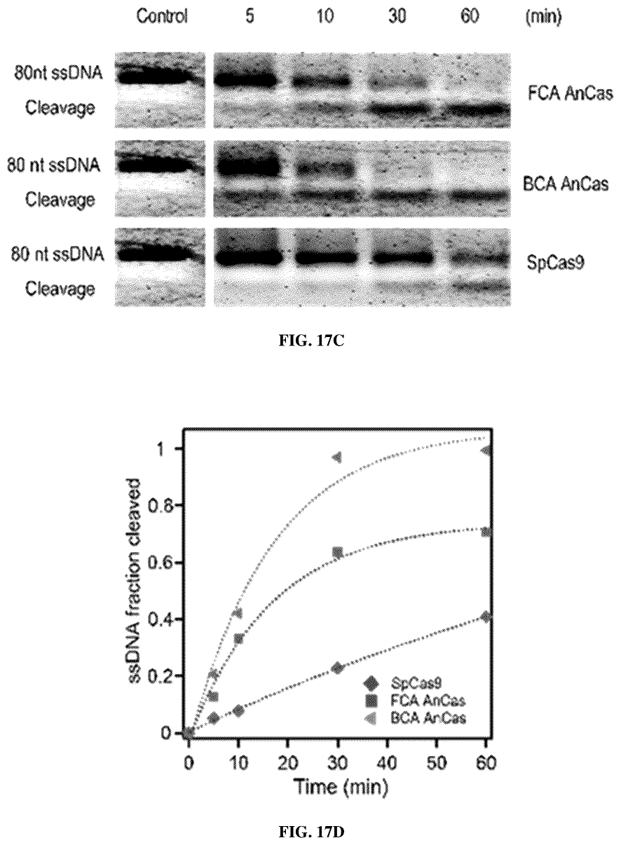

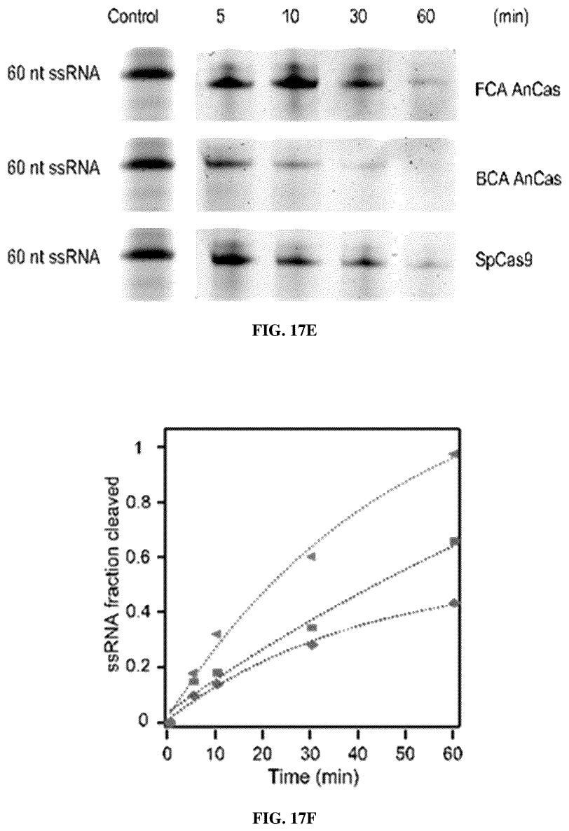

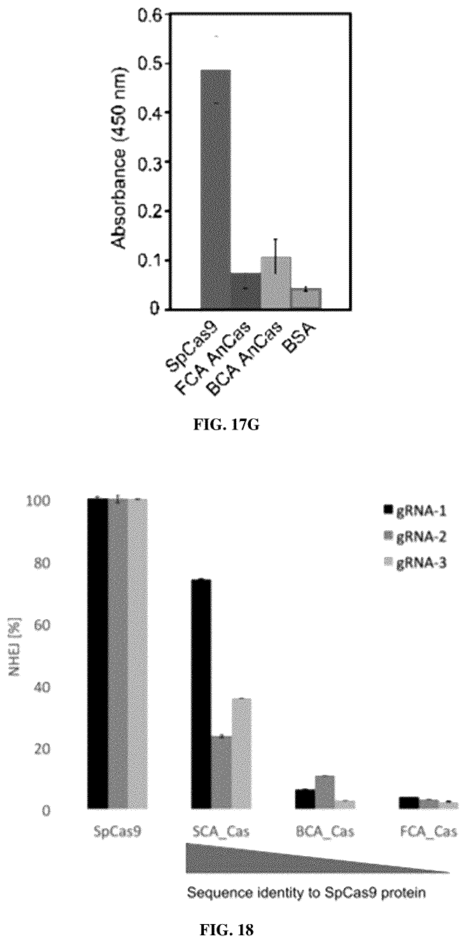

SpCas9 and the five AnCas were incubated for 10 minutes at 37° C. with a target plasmid DNA and TGG PAM recognition. From the agarose gel of cleavage products in FIG. 17A, it can be observed that, as expected, SpCas9 only linearized plasmid DNA when using its own sgRNA, although more efficiently when using the 20 nt spacer version, and sgRNAs from other species mostly resulted in nicked products leaving most supercoiled DNA substrate intact. On the contrary, LFCA Cas and LBCA Cas were able to nick and linearize plasmid DNA with all sgRNAs, the E. faecium sgRNA showing better efficiency for LFCA Cas, and the 18 nt sgRNA from S. pyogenes preferred for LBCA Cas. The other AnCas were also tested, observing that mostly LFCA Cas and LBCA Cas had a marked promiscuity for sgRNA. All other AnCas and SpCas9 seemed to work best with a 20 nt sgRNA from S. pyogenes (FIG. 17B).

Previous studies have indicated the contribution of the REC domain to sgRNA recognition specificity. As shown in Table 3, this domain exhibits the highest RMSD differences, with a decreasing RMSD trend from the oldest to the newest AnCas. These findings, along with the sgRNA promiscuity observed in oldest AnCas, suggest that evolutionary pressure might have guided Cas nucleases to an enhanced guide specificity over time. In fact, this promiscuity has been already observed in type II-C Cas9, which has been suggested to be an ancient reminiscence of Cas9 nucleases. In these nucleases, this promiscuity is also associated to PAM-independent ssDNA cleavage and weaker substrate DNA unwinding capabilities.

TABLE 3
RMSD values of different SpCas9 protein
domains obtained from AnCas

| | LFCA | LBCA | LSCA | LPCA | LPDCA |
|---|---|---|---|---|---|
| Domains | AnCas | AnCas | AnCas | AnCas | AnCas |
| RuvCI | 0.5820 | 0.3722 | 0.3414 | 0.3415 | 0.3617 |
| BH | 0.5877 | 0.3153 | 0.3453 | 0.3240 | 0.3272 |
| REC1 | 0.8105 | 0.5504 | 0.6405 | 0.5821 | 0.4768 |
| REC2 | 1.6140 | 0.8739 | 0.3722 | 0.5694 | 0.5194 |
| REC | 2.9309 | 1.1052 | 1.0937 | 0.9087 | 1.1426 |
| RUVC II | 0.8643 | 0.7294 | 0.6052 | 0.7951 | 0.7577 |
| HNH | 1.1783 | 0.8971 | 0.8710 | 0.9024 | 0.7063 |
| RUVC III | 2.1138 | 1.0338 | 1.0573 | 1.1097 | 0.9155 |
| PI | 1.9637 | 1.1284 | 1.0394 | 0.9443 | 0.7475 |

In another line of experiments, using the same in vitro plasmid cleavage assay as noted above, the ability of LFCA Cas to use sgRNAs with a targeting sequence linked to various tracrRNA sequences was investigated. TracrRNA sequences were employed corresponding to the tracrRNA components employed by Cas9 gRNAs of various existing bacterial species. Thus, a plasmid was provided including the S. pyogenes PAM TGG. It was incubated either with SpyCas9 or LFCA Cas in the presence of sgRNA having a tracrRNA derived from each of the bacterial species Streptococcus thermophilus, Enterococcus faceium, Clostridium perfringens and Finegoldia magna or a S. pyogenes sgRNA with normal 20 nt-spacer or shortened 18 nt-spacer. For further information on the gRNA sequences employed, reference may be made to Gasiunas et al., 2020 (Nat Commun. 11(1):5512; see supplementary data providing gRNA sequences of identified Cas9 orthologs). Following incubation, the reaction products were run on an agarose gel and the degree of supercoiled, nicked and linear DNA observed. The gel results are shown in FIG. 8. It is evident that LFCA Cas9 has very flexible gRNA use. It was able to nick or linearize the plasmid DNA regardless of the tracrRNA element of the gRNA employed. Indeed, improved cleavage was seen with some sgRNAs other than a conventional S. pyogenes sgRNA. Such gRNA flexibility is not shown for SpyCas9 and, as indicated above, is believed to be another novel property of LFCA Cas.

### Thermal and pH Stability

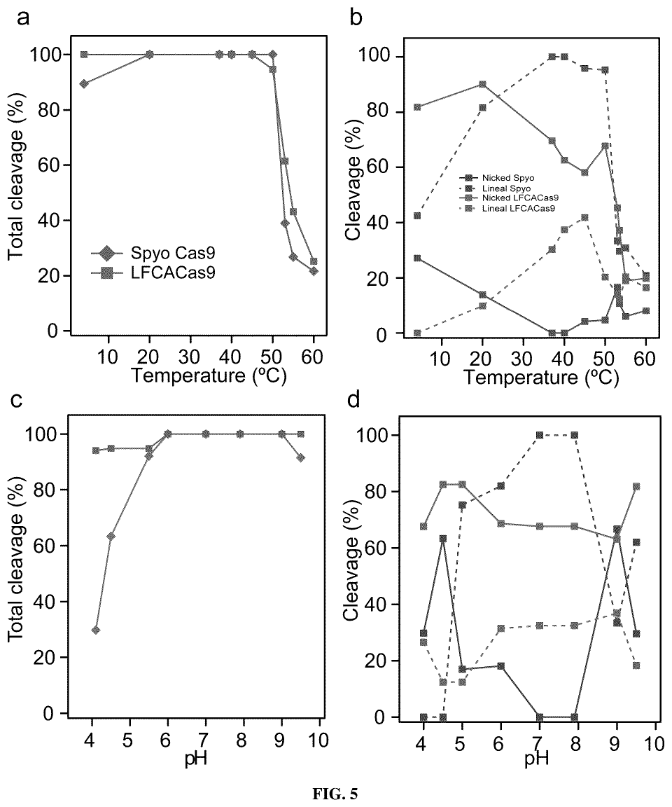

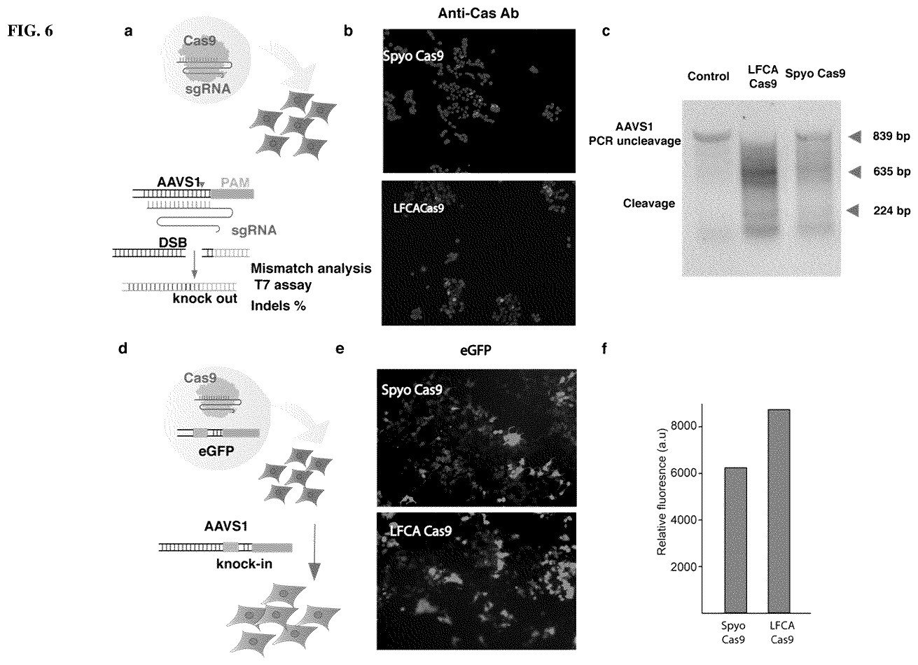

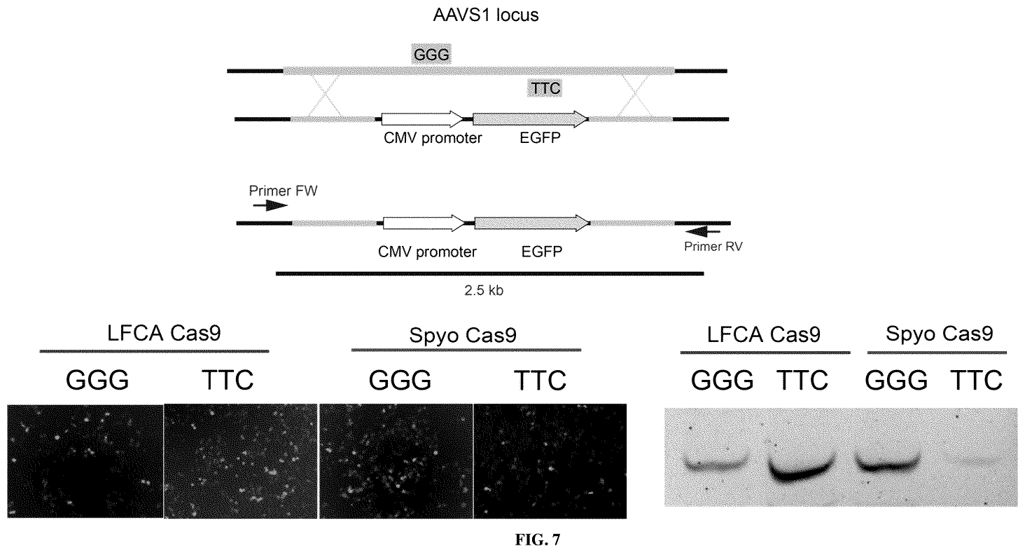

The thermal stability of LFCA Cas was studied by performing a cleavage reaction for 1 hour at pH 7.9 and at different temperatures ranging from 4° C. to 60° C. LFCA Cas showed higher activity than SpyCas9 at low temperatures (4° C. and 20° C.) and presented higher thermal stability from 53° C. to 60° C. (FIG. 5a). The nicking and endonuclease activities were calculated and it was observed that LFCA Cas had nicking activity at lower temperatures; at higher temperatures, the two activities were equally distributed (FIG. 5b).

For assessment of pH stability, the assay was performed at different pH values (from 4 to 9.5) at 37° C. (FIG. 5c). At acidic pH, LFCA Cas maintained higher activity in comparison to SpyCas9. At alkaline pH, the activity of LFCA Cas remained the same and SpyCas9 showed its optimal performance. As regards nicking and endonuclease activity, the pH affects the activities in a similar way as temperature (FIG. 5d). These results show the high stability that, in general, ancestral enzymes possess.

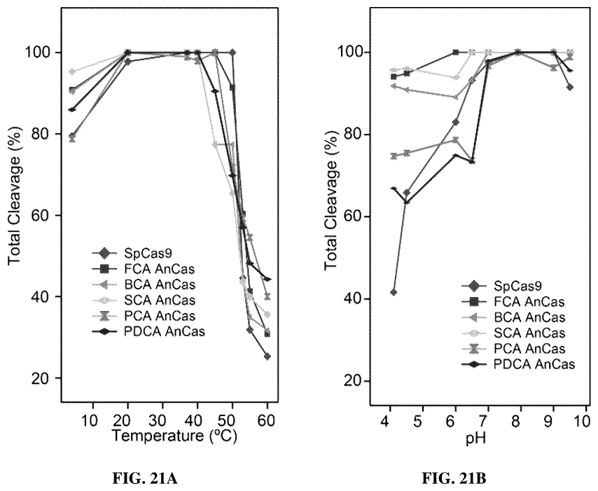

As mentioned above, AnCas, in particular LFCA AnCas, might share some commonalities with type V effector nucleases lacking a HNH domain (e.g., Cpf1 (Cas12a), Cas14 (Cas12f) or CasΦ (Cas12j)27-30). Given those numerous ancestral enzymes have demonstrated the ability to work in wider pH and temperature ranges, so indicating later adaptation to environmental conditions, the AnCas nucleases were tested in another line of experiments under different temperature and pH conditions. As shown in FIG. 21A-B, the oldest AnCas such as LFCA Cas, LBCA Cas and LSCA Cas showed high activity at pH values below 7, unlike SpCas9 and newer AnCas, where activity drops abruptly. In terms of temperature, AnCas endonucleases outperformed SpCas9 at low and high temperatures, below 10° C. and above 50° C.

### HEK293T Cell Genome Editing by LFCA Cas

HEK293T cells were transfected with an expression plasmid carrying an LFCA Cas humanized gene to study the ancestral enzyme effectiveness at editing genomic DNA. A gRNA to target the AAVS1 locus with S. pyogenes PAM was designed. The expression plasmid with encoded LFCA Cas was co-transfected with another plasmid to express the gRNA (FIG. 6a).

Then, the genomic DNA was extracted to study the insertion and deletion events (indels). Intracellular LFCA Cas expression was confirmed by making an immunofluorescent image of the cells using an anti-Cas9 antibody (orange) (FIG. 6b). The cell nucleus was dyed blue by DAPI. Cells were observed that expressed the LFCA Cas in the nucleus, in the same way as SpyCas9. The genomic DNA was extracted from the HEK293T cells after 72 hours of transfection and a fragment of the AAVS1 locus was amplified where the Cas enzyme cleavage was targeted.

A T7E1 endonuclease assay was performed with these fragments to confirm genome editing (FIG. 6c). After T7E1 incubation, the two expected fragments were observed, confirming indel formation after LFCA Cas transfection. The same was observed with intracellular expression of SpyCas9 as control.

In addition, knock-in activity after LFCA Cas and SpyCas9 genome cleavage was studied. The same strategy as in the previous experiment was followed, but a DNA template containing the eGFP gene flanked by homologous sequence to the AAVS1 locus was added to promote homologous end joining (HDR) (FIG. 6d). 72 hours after transfection, cells presented green fluorescence (FIG. 6e). The quantified fluorescence intensity showed higher values in the cells transfected with LFCA Cas (FIG. 6f).

A similar knock-in experiment targeting the AAVS1 region was carried out but using a different PAM than that of SpyCas9. In this experiment, the TTC PAM was targeted (FIG. 7). Cells were transfected with a gRNA and LFCA Cas or SpyCas9. Fluorescence was observed after 72 hours in all the samples from the DNA template (some transient fluorescence in the TTC sample with SpyCas9). The gDNA was extracted and the AAVS1 locus was amplified. The PCR amplicons were run on a gel. The expected band in all the LFCA Cas samples was observed but not in samples with SpyCas9. The gDNA was extracted and amplified at the AAVS1 locus. The PCRs amplicon were run on an electrophoresis gel and the expected band was observed in all the samples apart from the TTC PAM targeted with SpyCas9 as expected.

### Example 2: Endonuclease Activity on Single Strand DNA and Single Strand RNA

As mentioned above, the oldest AnCas (LFCA Cas and LBCA Cas) showed a remarkable nickase activity. This nickase activity may be related to ssDNA activity. ssDNA cutting activity has been suggested to be an ancestral trait present in smaller Cas9 such as subtype II-C Cas9. This could also be reflected in the nickase activity of the ancestral forms from subtype II-A, such as AnCas. Earlier forms of Cas9 with smaller catalytic domains might have been the origin of this ssDNA cutting activity that was still present in larger ancestral nucleases, which then gradually evolved towards DSB activity over time as part of a specialization process.

The activity of the ancestral Cas9 enzymes (LFCA Cas, LBCA Cas and LSCA Cas) on a single-stranded DNA was tested. The single-stranded plasmid m13mp18 linearized by EcoRI restriction enzyme was used as a substrate. The AnCas enzymes and SpyCas9 were each incubated with the plasmid and a gRNA designed to target the plasmid. As control, the DNA and enzyme (but no gRNA) were incubated together (FIG. 14). The three ancestral enzymes were found to cleave the single-stranded DNA with or without gRNA. A similar activity was seen with SpyCas9 when manganese was present in the reaction. The LSCA Cas showed the highest cleavage rate for the single-stranded DNA.

In another line of experiments, the oldest AnCas were tested with a 85 nt-ssDNA substrate containing a target sequence complementary to the 20 nt-spacer region of a Spy-sgRNA. As shown in FIGS. 17C and 17E, LFCA Cas and LBCA Cas showed highest levels of ssDNA cleavage than those of SpCas9. Exponential fits to the data showed much faster rates for LFCA Cas and LBCA Cas, reaching almost full cleavage for LBCA Cas (Table 4). Activity was tested on a 60 nt-ssRNA target showing comparable results (FIGS. 17D and 17F). Exponential fits of the cleavage activity revealed maximum rate and amplitude for LBCA Cas, reaching again full cleavage (Table 4). These results demonstrate that both ancient LFCA Cas and LBCA Cas, and in particular LBCA Cas, behave as RNA-guided RNAses.

The activity of the oldest AnCas on single-stranded substrates suggests that early Cas nucleases might have been active on those substrates, which seems to be an ancient trait, as mentioned before. These abilities may have additional important implication, given that the remarkable activity of LBCA Cas on ssDNA and ssRNA resembles that of Cas12a, Cas14 and Cas13a, which suggest a connection among the activities of all class 1 effector nucleases. This functional promiscuity also puts LBCA Cas forward as a highly versatile endonuclease for genome editing applications.

It was further investigated whether the oldest AnCas endonucleases, which display more promiscuous features, may also have a different response towards an anti-Cas9 antibody. LBCA Cas and LFCA Cas were incubated with an anti-Cas9 rabbit antibody. An ELISA test showed a diminished antibody binding (FIG. 17g). This would be expected given that host organisms carrying these nucleases have been long extinct and therefore have not been in contact with any living organisms. It can be reasoned that antibodies against Cas9 may have a weaker response towards ancient Cas forms. This lower antibody response might be of interest for potential applications in in vivo editing, where the immune response towards SpCas9 and other modern endonucleases represents a current limitation.

TABLE 4
Kinetics parameters of LFCA and LBCA AnCas and SpCas9 determined
from the single exponential decay curves in FIG. 17E and 17F.

| Kinetics | SpCas9 | | LFCA AnCas | | LBCA AnCas | |
|---|---|---|---|---|---|---|
| parameters | ssRNA | ssDNA | ssRNA | ssDNA | ssRNA | ssDNA |
| k_{cleave} (min⁻¹) | 0.016 ± 0.009 | 0.002 ± 0.008 | 0.002 ± 0.01 | 0.045 ± 0.01 | 0.014 ± 0.006 | 0.045 ± 0.02 |
| Max fraction | 0.43 | 0.41 | 0.66 | 0.70 | 0.97 | 0.99 |
| R² | 0.9856 | 0.9902 | 0.9807 | 0.9824 | 0.9954 | 0.9529 |

### Example 3: In Vivo Activity of AnCas Variants

The genome editing activity of these ancestral nucleases was tested in mammalian cells (HEK293T) in culture, to answer the question whether these synthetic ancestral Cas can perform DNA cleavage; double strand breaks (DSB), and trigger editing in cells by non-homologous end joining (NHEJ) under similar conditions as those associated with the standard SpCas9. The cells were co-transfected with plasmid vectors containing the humanized versions of AnCas or SpCas9, as well as the corresponding sgRNAs (standard sgRNA from S. pyogenes carrying a 20-nt spacer target with SEQ ID NO: 237 to 239).

Seventy-two hours after co-transfection, cells were collected and the genomic DNA extracted. In vitro site-specific editing was measured in the HEK293T cells by Next-generation Sequencing (NGS) using advanced analysis with Mosaic finder software.

As shown in FIG. 18, AnCas endonucleases performed robust gene editing in human genomic DNA, except for LFCA Cas. This was expectable given the unique features of LFCA Cas, which presumably does not use the HNH domain for cleavage and seems to work better in single-stranded substrates, akin to other types of Cas nucleases.

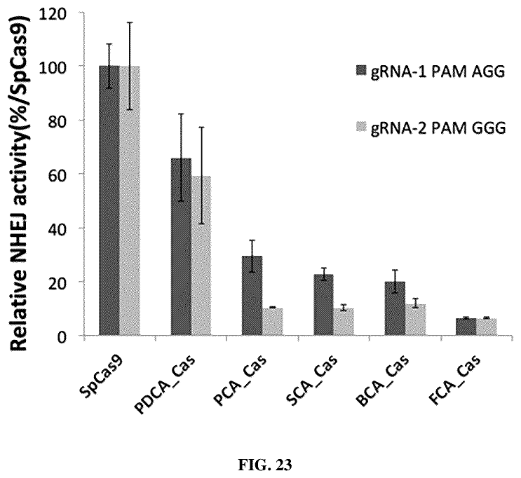

Site-specific cleavage was tested using a Traffic Light Reporter (TLR) based on RFP reconstitution. This method allows monitoring of DNA repair in HEK293T cells based on fluorescence activated cell sorting (FACS). Using again conditions optimized for SpCas9, the results were in line with those determined by NGS (FIG. 23), demonstrating their robustness.

### Materials and Methods

### Ancestral Sequence Reconstruction

The starting Cas9 sequences as noted above and listed in Table 1 and FIGS. 1 and 9 were downloaded from the NCBI database. Alignment of the sequences was performed using MUSCLE software on the MEGA platform and manually edited. The best evolutionary model was inferred using MEGA, resulting in the Jones-Tylor-Thornton (JTT) with gamma distribution model. Phylogeny was carried out using BEAST v1.8.4 package software including the BEAGLE library for parallel processing and based on Bayesian inference using Markov Chain Monte Carlo (MCMC). The divergence times were estimated by uncorrelated log-normal clock model (UCLN), using molecular information from TTOL with default birth and death rates. Calculations were run in a multicore server. From the generated trees, 25% of them were discarded as burn-in with the LogCombiner utility from BEAST. The MCMC log file was verified using TRACER and it was ensured that all parameters showed effective sample size (ESS)>100. Posterior probabilities of all nodes were above 0.65 and most of them were near 1. Figure tree v1.4.2 was used to visualize and edit the phylogenetic tree. Finally, ancestral sequence reconstruction was performed by maximum likelihood using PAML 4.8 with a gamma distribution for variable replacement rates across sites and the JTT model. Posterior probabilities were calculated for all amino acids and the residue with the highest posterior probability was chosen for each site. The Last Firmicute Common Ancestor (LFCA), Last Bacilli Common Ancestor (LBCA, Last Streptococci Common Ancestor (LSCA),

Last Pyogenic Common Ancestor (LPCA), Last Pyogenic-Dysgalactie Common Ancestor (LPDCA) were selected from the tree for reconstruction.

The node sequences identified by the method described above are set out in the sequence listing provided with this application which is expressly incorporated herein in its entirety.

### Protein Production and Purification

An LFCA Cas coding sequence was synthesized with codon optimization for E. coli cell expression. The coding sequence was cloned in a pBAD/His expression vector (ThermoFisher) and transformed in E. coli BL21 (DE3) (Life Technologies) for protein expression. A SpyCas9 expression plasmid was purchased from Addgene (Plasmid #62934). Cells were incubated in LB medium at 37° C. until OD600 reached 0.6. L-arabinose was added to 0.1% to cells for expression of LFCA Cas and IPTG was added to 1 mM to cells for expression of SpyCas9 with protein induction overnight at 20° C. Cells were pelleted by centrifugation at 4000 rpm. Pellets were resuspended in extraction buffer (20 mM HEPES pH 7.5, 300 mM NaCl, 25 mM Imidazole, 0.5 mM TCEP). 100 mg/ml of lysozyme (Thermo Scientific) was added to the pellet with incubation for 15 mins. Then the pellet was sonicated for 3 cycles for 10 mins at 30% amplitude. Cell debris was separated by ultracentrifugation at 33,000 g for 1 h. For purification, the supernatants were mixed with a His GraviTrap affinity column (GE Healthcare) and eluted with elution buffer (20 mM HEPES pH 7.5, 300 mM NaCl, 500 mM Imidazole, 0.5 mM TCEP). Proteins were further purified by size exclusion chromatography using a Superdex 200 HR column (GE Healthcare) and eluted in 20 mM HEPES pH 7.5, 1 M KCl, 10 mM MgCl₂, 0.5 mM TCEP. For protein purification verification, SDS-PAGE was used with 8% gels. The protein concentration was calculated by measuring the absorbance at 280 nm in Nanodrop 2000C.

gRNA Synthesis

gRNA with the complementary sequence to the target was synthesized and cloned into a pUC18 vector. gRNA sequence was amplified by PCR using Phusion® Hot Start Flex DNA Polymerase (NEB). PCR product was purified using mi-PCR Purification Kit (Metabion). gRNA was synthesized using HiScribe T7 High Yield RNA Synthesis Kit (NEB). The PCR fragment had the T7 promoter at the 5′ end and the sequence from sgRNA of S. pyogenes at the 3′ end. The reaction was incubated overnight and sgRNA was purified following the protocol of the kit Monarch® RNA Purification Columns. gRNA integrity was analysed by electrophoresis with 2% agarose gel with TBE buffer.

### In Vitro Cleavage Assay

In vitro cleavage assay was performed with purified LFCA Cas and SpyCas9. In all the assays, 30 nM Cas nuclease was incubated for 15 mins with 30 nM gRNA at 1:1 ratio in the cleavage buffer (100 mM NaCl, 50 mM Tris-HCl, 10 mM MgCl₂, 100 μg BSA, pH 7.9) at 37° C. Then, 3 nM target DNA was added and incubated for different times depending on the experiment. Reaction was stopped by adding 6× loading dye (NEB) with EDTA and the final reaction products run on a 2% agarose gel. Gels were dyed with SYBR gold (ThermoFisher) and imaged with ChemiDoc XRS+System (Bio-Rad). Cleavage was quantified by ImageJ.

### In Vitro Thermal and pH Stability.

The assay was performed following the previously explained protocol for in vitro cleavage but changing the conditions. For thermal stability, the assays were performed at pH 7.9 with the temperature varied from 4° to 60° C. For pH stability, the assays were performed at 37° C. and the pH was changed from 4 to 9.5. After 1 hour, the reaction was stopped by adding 6× loading dye (NEB) with EDTA and the final reaction products run on a 2% agarose gel. Gels were dyed with SYBR gold (ThermoFisher) and imaged with ChemiDoc XRS+System (Bio-Rad). Cleavage was quantified by imageJ.

### PAM Library Construction

A DNA library containing 7 random nucleotides was designed and cloned into a pUC18 plasmid by Genscript. This random library was transformed in XL1blue E. coli and amplified several times to achieve the maximal variability in the PAM sequences. A PCR fragment of 844 bp was amplified using the primers (F′ AATAGGCGTATCACGAGGC (SEQ ID NO: 6) and R′ AGCGAGTCAGTGAGCGAG (SEQ ID NO: 7) from the DNA library and containing the 7 random nucleotides.

### PAM Determination

PAM determination assay was performed by incubating in cleavage buffer: 3 nM of PCR fragment from the DNA library with 30 nM of LFCA Cas and 30 nM of gRNA targeting the 20 nucleotides upstream the 7 random nucleotides. The reaction was incubated for 1 hour at 37° C. Reaction was stopped by adding 6× loading dye (NEB) with EDTA and the final reaction products run on a 2% agarose gel. Gels were dyed with SYBR gold (ThermoFisher) and imaged with ChemiDoc XRS+System (Bio-Rad). The small fragment of 278 bp was purified from the agarose gel with GeneJet Gel Extraction kit (ThermoFisher). The fragment was sequenced by Ion Torrent and the obtained reads were mapped in the reference sequence. The reads that aligned to the reference with 0 mismatches were selected and the frequency was calculated for each PAM.

### HEK293T Cell Genome Editing

Cells were maintained in DMEN+10% FBS medium supplemented with 1% (w/v) L-glutamine and penicillin-streptomycin (100 IU/ml). Humanized coding sequences for LFCA Cas and SpyCas9 were cloned in a pCDNA 3.1 (ThermoFisher) expression vector and gRNA was cloned in a TOPO vector (ThermoFisher). Plasmids were incubated with lipofectamine LTX (ThermoFisher) for 5 minutes and co-transfected into cells. The medium was changed after 24 hours transfection and cells were collected after 72 hours. gDNA was extracted from cells using DNAzol Reagent (ThermoFisher) according to the manufacturer's protocol. DNA target was amplified by PCR using Phusion® Hot Start Flex DNA Polymerase (NEB) using primers (F′ TATTGTTCCTCCGTGCGTCAG (SEQ ID NO: 8) and R′GACGAGAAACACAGCCCCA (SEQ ID NO: 9)) from gDNA.

The T7EI assay was performed using as substrate these PCR amplicons to confirm indel formation. The T7E1 endonuclease (NEB) was used according to the manufacturer's protocol. Reaction was stopped by adding 6× loading dye (NEB) with EDTA and the final reaction products run on a 2% agarose gel. Gels were dyed with SYBR gold (ThermoFisher) and imaged with ChemiDoc XRS+System (Bio-Rad).

For knock-in experiments the same strategy was used, but a double stranded DNA template containing the eGFP gene and CMV promoter flanked by 500 bp homologous arms of the AAVS1 locus was utilised. After 72 hours the immunofluorescence was quantified by confocal microscopy.

### Immunofluorescence Studies

HEK293T cells were fixed with 4% paraformaldehyde for 30 mins after 24 hours of LFCA Cas and SpyCas9 plasmid transfection. Cells were incubated with 0.2% Triton X-100/PBS at RT for 30 mins and then incubated for 1 hour with 3% BSA, 0.05% Tween 20 for the blocking step. Cells were washed 3 times with TPBS (0.05% Tween-PBS) and incubated for 1 hour at 37° C. with a polyclonal anti-Cas9 antibody (1:100, 600-401-GK0. Thermofisher). Cells were washed 3 times with TPBS and incubated for 10 mins with a secondary antibody (goat anti-rabbit labelled with Alexa Fluor 555. 1:200, A-21428, Thermofisher). DAPI was added in this step and cells were washed one last time and visualized by confocal microscopy.

In Vitro Cleavage Assay for gRNA Promiscuity.

For in vitro cleavage for gRNA promiscuity, DNA plasmid carrying TGG PAM was used. The cleavage assay was performed in cleavage buffer (100 mM NaCl, 50 mM Tris-HCl, 10 mM MgCl₂, 100 μg/BSA, pH 7.9) at 37° C. 3 nM of AnCas and SpCas9 were incubated for 15 min with 3 nM sgRNA of each species at 1:1 ratio in cleavage buffer and 3 nM DNA plasmid was added. After 10 min, the reaction was stopped by adding 6× loading dye (NEB) with EDTA and run 2% agarose gel. Similarly, gels were dyed with SYBR gold (ThermoFisher Scientific) and imaged with ChemiDoc XRS+System (Bio-Rad). Cleavage was quantified by ImageJ.

In Vitro Cleavage Assay for ssDNA and ssRNA.

In vitro cleavage assay was performed with purified LFCA (FCA) AnCas, LBCA (BCA) AnCas and SpCas9 endonucleases. In all assays, 30 nM enzyme was incubated for 15 min with 30 nM sgRNA (Spy-sgRNA 20 nt) at 1:1 ratio in the cleavage buffer (100 mM NaCl, 50 mM Tris-HCl, 10 mM MgCl₂, 100 μg/BSA, pH 7.9) at 37° C. Then, 3 nM target (ssDNA or ssRNA) was added and incubated for different time intervals (0, 5, 10, 30 and 60 min). For ssDNA target, reaction was stopped by adding 6× loading dye (NEB) with urea. Samples were boiled for 10 min at 80° C. and were resolved by 2.5% denaturing urea agarose gel. For ssRNA target, reaction was stopped by adding 2×RNA gel-loading buffer (NEB) with urea. Samples were boiled for 10 min at 95° C. and were resolved by 15% denaturing urea polyacrylamide gel electrophoresis. In all cases, gels were dyed with SYBR gold (ThermoFisher Scientific) and imaged with ChemiDoc XRS+System (Bio-Rad). Cleavage was quantified by ImageJ and fitted with single-exponential decay curve.

### ELISA Test

Elisa test was performed by using a modified protocol described elsewhere⁶⁰. Briefly, 1 μg/well of SpCas9, LFCA AnCas, LBCA AnCas and bovine serum albumin (BSA, Sigma Aldrich) were diluted in 1× bicarbonate buffer and coated onto 96-well plates (ThermoFisher Scientific) overnight at 4° C. Plates were washed with 1× wash buffer (TBST, ThermoFisher Scientific) and blocking with 1% BSA blocking solution for 1 hour at room temperature. Anti-Cas9 rabbit antibody (Rockland, 600-401-GK0) was diluted 1:25000 in 1% BSA blocking solution and plates were incubated for 2 hours at room temperature. Then, plates were washed and HRP-conjugated goat anti-Rabbit IgG (H+L) (Invitrogen), diluted 1:2000 in 1% BSA blocking solution, was added, and incubated for 1 hour at room temperature. Finally, 3,3′,5,5′-Tetramethylbenzidine ELISA substrate solution (ThermoFisher Scientific) was added and incubated for 10 min at room temperature. The reaction was stopped with 1 N sulfuric acid. The absorbance was measured at 450 nm by using a VICTOR X5 microplate reader (PerkinElmer).

### Human HEK293T Cells In Vivo Cleavage.

Functional validation of ancestral Cas nucleases was carried out in human HEK293T cells, as described elsewhere (Harms, D. W. et al. Human Genetics 83, 2014). Cells were grown in DMEM medium (Dulbecco's Modified Eagle Medium, Gibco), supplemented with sterile-filtered 10% fetal bovine serum (FBS), 10 mM HEPES pH 7.4, 2 mM L-glutamine and penicillin (100 IU/ml)-streptomycin (100 μg/ml) and handled under aseptic conditions using a sterile hood. HEK293T cells were cultured in incubators at 37ºC, 95% humidity and 5% CO₂. Humanized AnCas were cloned into pcDNA3.1 plasmid expression vector (ThermoFisher). The sgRNA target sequences were designed with Breaking-Cas web tool⁶² and cloned into MLM3636 plasmid vector (Addgene #43860) through Golden Gate cloning method. SpCas9 from hCas9 plasmid (Addgene #41815) was used as a positive control. For the in vivo genome-editing tests, cells were plated in 24-well plates at a density of 4×10⁵ cells/ml in a 0.5 ml volume of DMEM without antibiotics. To these cells, 1 μg of hCas/hAnCas plasmid and 0.5 μg of the corresponding sgRNA plasmid were transfected with 2 μl of Lipofectamine 2000 (Life Technologies) diluted in 100 μl of Opti-MEM (Gibco) per well. 72 hours post-transfection genomic DNA was isolated with High Pure Template Preparation Kit (Roche). INDEL occurrence was assessed by T7 Endonuclease I assay on PCR-amplified DNA fragments surrounding the target DSB.

Reconstructed Ancestral Sequences with Node Identification According to FIG. 1.

All are 1340 or 1368 amino acid residues (depending on the reconstruction method). Sequences are disclosed as set forth in SEQ ID NOs: 1 to 5 and 10 to 236.

SEQ ID NOs: 1 to 5 correspond to the ancestral Cas proteins exemplified above.

1-24. (canceled)

25. A Cas nuclease comprising or consisting of:
(i) the LFCA nuclease having the amino acid sequence set forth in SEQ ID NO: 1;
(ii) the LBCA nuclease having the amino acid sequence set forth in SEQ ID NO: 2;
(iii) the LSCA nuclease having the amino acid sequence set forth in SEQ ID NO: 3;
(iv) the LPCA nuclease having the amino acid sequence set forth in SEQ ID NO: 4;
(v) the LPDCA nuclease having the amino acid sequence set forth in SEQ ID NO: 5; or
(vi) a variant of the Cas nuclease according to any one of (i)-(v), wherein said variant shares:
at least 60% of sequence identity with the amino acid sequences of SEQ ID NO: 1, or
at least 75% of sequence identity with the amino acid sequences of SEQ ID NO: 2, or
at least 80% of sequence identity with the amino acid sequences of SEQ ID NO: 3, or
at least 85% of sequence identity with the amino acid sequences of SEQ ID NO: 4, or
at least 95% of sequence identity with the amino acid sequences of SEQ ID NO: 5, and
further wherein said variant retains one or several of the following distinguishing characteristics as compared to SpyCas9:
(a) a higher percentage of nicked DNA plasmid template and/or a lower percentage of linearized DNA plasmid template under conditions whereby SpyCas9 results in substantially exclusively linearized DNA plasmid template;
(b) a higher nick rate and/or a lower linearization rate on a DNA plasmid target under conditions whereby SpyCas9 results substantially exclusively in linearization (a ratio of linearized DNA plasmid target to nicked DNA plasmid template of at least about 4:1) while the variant provides observable nicked target;
(c) a relaxed PAM requirement comparable to any of LFCA, LBCA or LSCA;
(d) the ability to cleave single-stranded DNA; and
(e) the ability to use a sgRNA in which the targeting sequence is linked to a tracrRNA component, wherein said tracrRNA component is selectable from the tracrRNA components of Cas9 gRNAs employed by a plurality of existing bacterial species.

26. The Cas nuclease according to claim 25, wherein said Cas nuclease has been modified by catalytic site mutagenesis to retain only its nickase activity.

27. The Cas nuclease according to claim 25, wherein said Cas nuclease comprises one or several amino acid changes by way of substitution or deletion, whereby the endonuclease and/or nickase activity of the Cas nuclease is retained and whereby the relaxed PAM specificity of LFCA Cas is retained.

28. The Cas nuclease according to claim 25, wherein said Cas nuclease comprises one or several conservative substitutions, whereby the endonuclease and/or nickase activity of the Cas nuclease is retained and whereby the relaxed PAM specificity of LFCA Cas is retained.

29. The Cas nuclease according to claim 25, wherein said Cas nuclease has been modified by catalytic site mutagenesis to abolish its nuclease activity.

30. The Cas nuclease according to claim 25, wherein said Cas nuclease is linked or fused with a non-nuclease effector of genetic modification or regulation.

31. A method of modifying or regulating a target nucleic acid sequence, the method comprising contacting the target nucleic acid sequence with (i) a Cas nuclease according to claim 25, and (ii) a guide RNA, wherein said guide RNA targets the Cas nuclease to the target sequence, further wherein either:
(a) said contacting is in vitro with an isolated target nucleic acid or in a cell ex vivo, with the proviso that the method is not a method of modifying the germ line identity of a human being; or
(b) the method is not a method of medical treatment practiced on the human or animal body.

32. The method according to claim 31, wherein said target nucleic acid sequence is a target DNA sequence in ex vivo human or animal cells.

33. A method of phylogenetic ancestral reconstruction for obtaining a functional, single effector Cas protein nuclease comprising:
(a) providing a phylogenetic tree from sequence analysis of a population of Cas sequences comprising naturally-occurring single effector Cas nuclease sequences of the same classification type and derived from a plurality of existing species;
(b) selecting an ancestral variant sequence by tracing back an evolutionary route from the phylogenetic tree, wherein the highest probability amino acid for each amino acid of the selected ancestral variant is determined, and
(c) producing said variant, wherein said variant is capable of exhibiting Cas protein endonuclease and/or nickase activity.

34. The method according to claim 33, wherein step (b) comprises compiling sequences of ancestral variants which are each just ancestral variants for a plurality of species' sequences forming a proportion of the sequences of the same genus.

35. The method according to claim 34, further comprising using said sequences to compile one or more ancestor variant sequences which are assigned as a genus ancestor and/or one or more ancestor variant sequences which are assigned as a class ancestor able to trace back to sequences of starting species of a plurality of genera.

36. The method according to claim 35, further comprising compiling at least one inter-class ancestor sequence able to trace back to starting species of more than one class.

37. The method according to claim 33, wherein said selected ancestral variant sequence equates with an evolutionary period of at least 500 million years from the present.

38. The method according to claim 33, wherein said selected ancestral variant sequence is an ancestral variant of Cas9 sequences of existing bacterial species.

39. The method according to claim 38, wherein the starting population of Cas9 sequences comprises a plurality of bacterial Cas9 sequences selected from two or more of Streptococcus, Enterococcus, Listeria, Clostridium, Pelagirhabdus, Halolactibacillus, Floricoccus, Vagococcus, Urinacoccus, Vagococcus, Dorea, Ruminococcus, Lachnospira, Anaerostipes, Oisenella and Bifidobacterium.

40. The method according to claim 38, wherein the starting population of sequences spans more than one bacterial class.

41. The method according to claim 33, wherein the selected ancestral variant sequence is an inter-class ancestral variant sequence able to trace back to starting species of more than one class.

42. The method according to claim 33, wherein the selected ancestral variant is determined to be capable of exhibiting endonuclease double strand DNA cleavage and is further converted to either a nickase only or a deadCas with no nuclease activity and/or is linked with a non-nuclease effector, e.g., in a fusion protein.

43. The method according to claim 38, wherein the ancestral variant of Cas9 sequences has one or several of the following characteristics:
(a) a ratio of linearized DNA plasmid target to nicked DNA plasmid template of between at least about 2.3:1 to at least 1:4 under conditions whereby SpyCas9 results in a ratio of linearized DNA plasmid target to nicked DNA plasmid template of at least about 4:1;
(b) relaxed PAM requirement comparable to any of LFCA, LBCA or LSCA;
(c) ability to cleave single-stranded DNA;
(d) ability to use a sgRNA in which the targeting sequence is linked to a tracrRNA component, wherein said tracrRNA component is selectable from the tracrRNA components of Cas9 gRNAs employed by a plurality of existing bacterial species.

44. The method according to claim 43, wherein the selected ancestral variant is further converted to a variant which is either a nickase only or a deadCas with no nuclease activity and/or provides linkage with a non-nuclease effector, e.g., in a fusion protein.
